(19) United States
(12) Patent Application Publication
(10) Pub. No.: US 2013/0202579 A1
(43) Pub. Date: Aug. 8, 2013

(54) USE OF CHOLINE-PHOSPHATE CYTIDYLYLTRANSFERASE-ALPHA (CCT-ALPHA) AS A BIOMARKER FOR CANCER PROGNOSIS

(71) Applicant: University of Pittsburgh - Of the Commonwealth System of Higher Education, Pittsburgh, PA (US)

(72) Inventors: Laura J. Niedernhofer, Jupiter, FL (US); Nikhil Ramchandra Bhagwat, Pittsburgh, PA (US); Alec E. Vaezi, Pittsburgh, PA (US)

(73) Assignee: University of Pittsburgh - Of the Commonwealth System of Higher Education

(21) Appl. No.: 13/758,780

(22) Filed: Feb. 4, 2013

Related U.S. Application Data
(60) Provisional application No. 61/594,540, filed on Feb. 3, 2012.

(51) Int. Cl. G01N 33/68

(52) U.S. Cl. CPC G01N 33/6893
USPC 424/94.6; 435/7.4; 435/6.13; 435/6.12; 506/9; 435/6.11

(57) ABSTRACT

It has previously been demonstrated that positive 8F1 immunohistochemistry is an indicator of a poor cancer prognosis and poor response to chemotherapy. The 8F1 antibody was generated by immunization against ERCC1, a DNA repair protein, but recognizes a second, previously unidentified protein in cells and tissues. Disclosed herein is the finding that in addition to ERCC1, the 8F1 antibody recognizes the choline phosphate cytidylyltransferase-α (CCTα) protein. Thus, provided herein is a method of determining the prognosis of a patient with cancer by specifically detecting expression of CCTα in a sample obtained from the subject. Also provided is a method of predicting the response of a cancer patient to treatment with a genotoxic therapy by specifically detecting expression of CCTα in a sample obtained from the subject.

### CROSS REFERENCE TO RELATED APPLICATIONS

This application claims the benefit of U.S. Provisional Application No. 61/594,540, filed Feb. 3, 2012, which is herein incorporated by reference in its entirety.

### ACKNOWLEDGMENT OF GOVERNMENT SUPPORT

This invention was made with government support under grant number ES016114 awarded by the National Institutes of Health. The government has certain rights in the invention.

### FIELD

This disclosure concerns the identification of choline-phosphate cytidylyltransferase alpha (CCTα) as a biomarker for cancer prognosis and response to therapy.

### BACKGROUND

The mouse monoclonal antibody 8F1 was raised against the human excision repair cross-complementation group 1 (ERCC1) protein. ERCC1 is a DNA repair protein required for nucleotide excision repair of damaged DNA. ERCC1 plays an important role in DNA repair in response to damage caused by platinum-based chemotherapeutics. A number of clinical trials have established a correlation between positive 8F1 immunohistochemistry (IHC) and a poor response to chemotherapy and a poor prognosis. For example, Olaussen et al. (N Engl J Med 355(10):983-991, 2006) reported an improvement in overall survival for adjuvant cisplatin chemotherapy in completely resected non-small cell lung carcinoma (NSCLC) patients with ERCC1 negative (negative 8F1 IHC) tumors, and no benefit for those patients with ERCC1 positive (positive 8F1 IHC) expression. Positive 8F1 IHC has also been linked to a poor prognosis for gastric cancer and esophageal cancer. Kwon et al. (Ann Oncol 18:504-509, 2007) determined that gastric cancer patients with negative ERCC1 expression exhibited an enhanced response to chemotherapy and better overall survival. ERCC1 expression, again as measured by 8F1 positive IHC, has also been linked to improved outcomes in patients with head and neck squamous cell carcinoma (HNSCC) treated with cisplatin. Handra-Luca et al. (Clin Cancer Res 13:3855-3859, 2007) found that HNSCC patients with low ERCC1 expression had a 4-fold increase in response to cisplatin-based chemotherapy and a lower risk of disease related death. These data suggest that 8F1 signal has predictive value in several tumor types. A study of patients with esophageal cancer treated with chemoradiation demonstrated that patients with ERCC1 negative biopsies showed a tendency toward prolonged overall survival and event free survival (Kim et al., Eur J Cancer 44(1):54-60, 2008). In contrast, in patients with NSCLC treated with surgery only, high expression of ERCC1, as measured by 8F1 immunohistochemical detection of ERCC1 in tumors, correlated with prolonged disease-free survival (Zheng et al., N Engl J Med 356(8): 800-808, 2007). This indicates that 8F1 signal also has prognostic value.

It has previously been reported that the 8F1 antibody recognizes a second, unknown protein in cells and tissues; the antibody is unable to distinguish between ERCC1-positive cells and ERCC1-deficient cells by IHC (Bhagwat et al., Cancer Res 69:6831-6838, 2009; Niedernhofer et al., N Eng J. Med. 356:2538-2541, 2007). Given the established link between positive 8F1 IHC and poor cancer prognosis and poor response to chemotherapy, it is desirable to identify the second protein recognized by the 8F1 antibody.

### SUMMARY

It has previously been demonstrated that positive 8F1 immunohistochemistry is an indicator of cancer prognosis and response to chemotherapy. Disclosed herein is the finding that in addition to ERCC1, the 8F1 antibody recognizes the choline phosphate cytidylyltransferase-α (CCTα) protein.

Provided herein is a method of determining the prognosis of a patient with cancer by specifically detecting expression of CCTα in a sample obtained from the subject. An increase in expression of CCTα in the sample compared to a control indicates a good prognosis for the patient.

Also provided is a method of predicting the response of a cancer patient to treatment with a genotoxic therapy by specifically detecting expression of CCTα in a sample obtained from the subject. An increase in expression of CCTα in the sample compared to a control predicts a poor response to the genotoxic agent.

The foregoing and other objects, features, and advantages of the invention will become more apparent from the following detailed description, which proceeds with reference to the accompanying figures.

### BRIEF DESCRIPTION OF THE FIGURES

FIGS. 1A-1E: CCTα is the second antigen recognized by 8F1. Whole cell lysate from the A2780 cell line was subjected to immunoprecipitation with 8F1 or D-10, an antibody specific for ERCC1. (FIG. 1A) Precipitated proteins were resolved by electrophoresis and visualized by gel silver staining. 8F1 precipitated two proteins (Band 1 and Band 2) migrating at a molecular size near ERCC1 (37 kD), while D-10 precipitates only the lower band. (FIG. 1B) Mass spectrometry analysis for Band 1 and Band 2. Shown are the two most frequent hits for each analysis. For 8F1, the top band (Band 2) was mostly composed of CCTα, while the bottom contained CCTα and ERCC1. For D-10, CCTα was not detected in analysis of the Band 1 or the region of the gel equivalent to Band 2. (FIG. 1C) Immunoblot detection of GFP-tagged CCTα stably overexpressed in HeLa cells. 8F1 (left) recognizes the recombinant protein and trace amounts of endogenous CCTα. A commercially available CCTα antibody specifically detects the recombinant protein. (FIG. 1D) Immunoblots of lysates from nine clones from A2780 stably transfected with a plasmid expressing shRNA against CCTα. An antibody against CCTα (middle panel) reveals that CCTα expression is knocked-down to a variable extent in the different clones. The same is detected with the antibody 8F1 (top panel), while the bottom band (ERCC1) is unaffected. Tubulin is used as a loading control (bottom panel). (FIG. 1E) Representative immunofluorescence images of CCTα knock-down clones (18-3 and 20-2) and control (A2780) cells immunostained with 8F1 and antibodies specific for CCTα and ERCC1 (FL297). CCTα knock-down abrogated 8F1 nuclear signal (star), but ERCC1 levels remained unchanged (arrow head).

FIGS. 2A-2B: The immunohistochemical nuclear signal of 8F1 is affected by CCTα expression. (FIG. 2A) Pellets of A2780 cells and the CCTα knock-down clone 20-2 were fixed, paraffin embedded and sectioned for immunohistochemistry. Immunostaining with anti-CCTα (top row) revealed nuclear stain only in A2780 cells. Hematoxylin is used as the counterstain. Immunostaining with 8F1 (middle row) revealed a diminished nuclear signal in the knock-down cells compared to A2780, indicating that 8F1 IHC signal is affected by CCTα expression. Immunostaining with the ERCC1-specific antibody EP2143 (bottom row) revealed equivalent nuclear signal in A2780 and the knock-down cell line, indicating that ERCC1 expression is not affected by CCTα shRNA. (FIG. 2B) Immunostaining of paraffin sections of a human dermal fibroblast pellet (ERCC1 WT) and a pellet of XP2YO cells (ERCC1-deficient from and XP-F patient) with EP2143Y antibody. The nuclear signal is apparent only in the normal fibroblasts, where the mutant cells show pancellular background staining.

FIGS. 3A-3D: 8F1 IHC signal in NSCLC depends on ERCC1 and CCTα expression levels. Graphical representation of the correlation between the nuclear signal intensity of a NSCLC tumor microarray (n=187) measured using AQUA. Each dot represents one tumor. The position of the dot is determined by the IHC intensity of the respective antibodies on the x and y axes. The solid line represents a polynomial linear regression. Note the higher degree of correlation (greater slope) between the two ERCC1 specific antibodies (FIG. 3A) than between other pairs of antibodies. 8F1 IHC signal had an equivalent correlation with ERCC1-specific signal (FIGS. 3B and 3C) and CCTα (FIG. 3D). R value is indicated in the upper left corner of each plot.

FIGS. 4A-4B: CCTα expression correlates with increased overall and disease-free survival. (FIG. 4A) Kaplan-Meier survival estimate for disease-free survival in NSCLC (n=86). (FIG. 4B) Kaplan-Meier survival estimate for recurrence-free survival for HNSCC. p value (log rank) is indicated.

FIGS. 5A-5B: CCTα does not contribute to 8F1 signal in AC. Conditional regression plots for the contribution of ERCC1 (FIG. 5A) or CCTα (FIG. 5B) to Log(8F1) intensity in the NSCLC cohort (n=187). ERCC1 (FL297) contribution to 8F1 staining is similar for the three histological types as the curves are similar (FIG. 5A). However, CCTα contributes to the 8F1 signal only in squamous cell carcinoma (SCC) and large cell carcinoma (LCC) but not in adenocarcinoma (AC) as demonstrated by a flat curve for this histology (arrow, FIG. 5B).

FIG. 6: Survival estimate of the NSCLC cohort stratified by T stage. Shown is a Kaplan-Meier survival estimate of the whole NSCLC cohort (n=187), stratified by T stage. P value (log rank); * indicates statistical significance.

### DETAILED DESCRIPTION

### I. Abbreviations

AC adenocarcinoma
CCTα choline-phosphate cytidylyltransferase alpha
CMV cytomegalovirus
ELISA enzyme-linked immunosorbent assay
ERCC1 excision repair cross-complementation group 1
FACS fluorescence activated cell sorting
FFPE formalin fixed and paraffin embedded
GFP green fluorescent protein
HNSCC head and neck squamous cell carcinoma
IF immunofluorescence
IHC immunohistochemistry
IP immunoprecipitate(d)
LCC large cell carcinoma
NSCLC non-small cell lung carcinoma
SCC squamous cell carcinoma
shRNA short hairpin RNA
WB Western blot
WCE whole cell extract

### II. Terms and Methods

Unless otherwise noted, technical terms are used according to conventional usage. Definitions of common terms in molecular biology may be found in Benjamin Lewin, Genes V, published by Oxford University Press, 1994 (ISBN 0-19-854287-9); Kendrew et al. (eds.), The Encyclopedia of Molecular Biology, published by Blackwell Science Ltd., 1994 (ISBN 0-632-02182-9); and Robert A. Meyers (ed.), Molecular Biology and Biotechnology: a Comprehensive Desk Reference, published by VCH Publishers, Inc., 1995 (ISBN 1-56081-569-8).

In order to facilitate review of the various embodiments of the disclosure, the following explanations of specific terms are provided:

Aerodigestive tract: The combined organs and tissues of the respiratory tract and the upper part of the digestive tract (including the lips, mouth, tongue, nose, throat, vocal cords, and part of the esophagus and windpipe). In the context of the present disclosure, cancers of the “upper aerodigestive tract” include, but are not limited to, head and neck squamous cell carcinoma, oral carcinoma, gastric carcinoma, esophageal carcinoma and nasopharyngeal cancer; and cancers of the lip, tongue, major salivary glands, gums and adjacent oral cavity tissues, floor of the mouth, tonsils, oropharynx, nasopharynx, hypopharynx, nasal cavity, accessory sinuses, middle ear, and larynx.

Antibody: A polypeptide ligand comprising at least a light chain or heavy chain immunoglobulin variable region which specifically recognizes and binds an epitope of an antigen. Antibodies are composed of a heavy and a light chain, each of which has a variable region, termed the variable heavy (V_{H}) region and the variable light (V_{L}) region. Together, the V_{H} region and the V_{L} region are responsible for binding the antigen recognized by the antibody.

Antibodies include intact immunoglobulins and the variants and portions of antibodies well known in the art, such as Fab fragments, Fab′ fragments, F(ab)′₂ fragments, single chain Fv proteins (“scFv”), and disulfide stabilized Fv proteins (“dsFv”). A scFv protein is a fusion protein in which a light chain variable region of an immunoglobulin and a heavy chain variable region of an immunoglobulin are bound by a linker, while in dsFvs, the chains have been mutated to introduce a disulfide bond to stabilize the association of the chains. The term also includes genetically engineered forms such as chimeric antibodies (for example, humanized murine antibodies), heteroconjugate antibodies (such as, bispecific antibodies). See also, Pierce Catalog and Handbook, 1994-1995 (Pierce Chemical Co., Rockford, Ill.); Kuby, J., Immunology, 3^{rd} Ed., W. H. Freeman & Co., New York, 1997.

Typically, a naturally occurring immunoglobulin has heavy (H) chains and light (L) chains interconnected by disulfide bonds. There are two types of light chain, lambda (λ) and kappa (k). There are five main heavy chain classes (or isotypes) which determine the functional activity of an antibody molecule: IgM, IgD, IgG, IgA and IgE.

References to “V_{H}” or “VH” refer to the variable region of an immunoglobulin heavy chain, including that of an Fv, scFv, dsFv or Fab. References to “V_{L}” or “VL” refer to the variable region of an immunoglobulin light chain, including that of an Fv, scFv, dsFv or Fab.

A “monoclonal antibody” is an antibody produced by a single clone of B-lymphocytes or by a cell into which the light and heavy chain genes of a single antibody have been transfected. Monoclonal antibodies are produced by methods known to those of skill in the art, for instance by making hybrid antibody-forming cells from a fusion of myeloma cells with immune spleen cells. Monoclonal antibodies include humanized monoclonal antibodies.

A “chimeric antibody” has framework residues from one species, such as human, and CDRs (which generally confer antigen binding) from another species, such as a murine antibody.

A “humanized” immunoglobulin is an immunoglobulin including a human framework region and one or more CDRs from a non-human (for example a mouse, rat, or synthetic) immunoglobulin. The non-human immunoglobulin providing the CDRs is termed a “donor,” and the human immunoglobulin providing the framework is termed an “acceptor.” In one embodiment, all the CDRs are from the donor immunoglobulin in a humanized immunoglobulin. Constant regions need not be present, but if they are, they must be substantially identical to human immunoglobulin constant regions, i.e., at least about 85-90%, such as about 95% or more identical. Hence, all parts of a humanized immunoglobulin, except possibly the CDRs, are substantially identical to corresponding parts of natural human immunoglobulin sequences. A “humanized antibody” is an antibody comprising a humanized light chain and a humanized heavy chain immunoglobulin. A humanized antibody binds to the same antigen as the donor antibody that provides the CDRs. The acceptor framework of a humanized immunoglobulin or antibody may have a limited number of substitutions by amino acids taken from the donor framework. Humanized or other monoclonal antibodies can have additional conservative amino acid substitutions which have substantially no effect on antigen binding or other immunoglobulin functions. Humanized immunoglobulins can be constructed by means of genetic engineering (see for example, U.S. Pat. No. 5,585,089).

A “human” antibody (also called a “fully human” antibody) is an antibody that includes human framework regions and all of the CDRs from a human immunoglobulin. In one example, the framework and the CDRs are from the same originating human heavy and/or light chain amino acid sequence. However, frameworks from one human antibody can be engineered to include CDRs from a different human antibody. All parts of a human immunoglobulin are substantially identical to corresponding parts of natural human immunoglobulin sequences.

Cancer, neoplasia, malignancy, and tumor: A neoplasm is an abnormal growth of tissue or cells that results from excessive cell division. Neoplastic growth can produce a tumor. The amount of a tumor in an individual is the “tumor burden” which can be measured as the number, volume, or weight of the tumor. A tumor that does not metastasize is referred to as “benign.” A tumor that invades the surrounding tissue and/or can metastasize is referred to as “malignant.” Malignant tumors are also referred to as “cancer.”

Hematologic cancers are cancers of the blood or bone marrow. Examples of hematological (or hematogenous) cancers include leukemias, including acute leukemias (such as acute lymphocytic leukemia, acute myelocytic leukemia, acute myelogenous leukemia and myeloblastic, promyelocytic, myelomonocytic, monocytic and erythroleukemia), chronic leukemias (such as chronic myelocytic (granulocytic) leukemia, chronic myelogenous leukemia, and chronic lymphocytic leukemia), polycythemia vera, lymphoma, Hodgkin's disease, non-Hodgkin's lymphoma (indolent and high grade forms), multiple myeloma, Waldenstrom's macroglobulinemia, heavy chain disease, myelodysplastic syndrome, hairy cell leukemia and myelodysplasia. In some cases, lymphomas are considered solid tumors.

Solid tumors are abnormal masses of tissue that usually do not contain cysts or liquid areas. Solid tumors can be benign or malignant. Different types of solid tumors are named for the type of cells that form them (such as sarcomas, carcinomas, and lymphomas). Examples of solid tumors, such as sarcomas and carcinomas, include fibrosarcoma, myxosarcoma, liposarcoma, chondrosarcoma, osteosarcoma, and other sarcomas, synovioma, mesothelioma, Ewing's tumor, leiomyosarcoma, rhabdomyosarcoma, colon carcinoma, lymphoid malignancy, pancreatic cancer, breast cancer, lung cancers, ovarian cancer, prostate cancer, hepatocellular carcinoma, squamous cell carcinoma, basal cell carcinoma, adenocarcinoma, sweat gland carcinoma, medullary thyroid carcinoma, papillary thyroid carcinoma, pheochromocytomas sebaceous gland carcinoma, papillary carcinoma, human papilloma virus (HPV)-infected neoplasias, papillary adenocarcinomas, medullary carcinoma, bronchogenic carcinoma, renal cell carcinoma, hepatoma, bile duct carcinoma, choriocarcinoma, Wilms' tumor, cervical cancer, testicular tumor, seminoma, bladder carcinoma, melanoma, and CNS tumors (such as a glioma (such as brainstem glioma and mixed gliomas), glioblastoma (also known as glioblastoma multiforme) astrocytoma, CNS lymphoma, germinoma, medulloblastoma, Schwannoma craniopharyogioma, ependymoma, pinealoma, hemangioblastoma, acoustic neuroma, oligodendroglioma, menangioma, neuroblastoma, retinoblastoma and brain metastasis).

In some embodiments of the methods disclosed herein, the solid tumor is non-small cell lung carcinoma, gastric carcinoma, esophageal carcinoma, pancreatic cancer, colon cancer, breast cancer, brain cancer, head and neck squamous cell carcinoma, squamous cell carcinoma of the lung, pulmonary papillary adenocarcinoma, mesothelioma, esophageal cancer, nasopharyngeal cancer, prostate cancer, adrenocortical carcinoma, cutaneous neuroendocrine cancer, gallbladder cancer, bile duct cancer, cervical cancer, serous ovarian cancer, epithelial ovarian cancer, endometrial cancer, bladder cancer, urothelial carcinoma, Ewing sarcoma, testicular cancer, neuroendocrine cancer, liver cancer, hepatocellular carcinoma, pituitary cancer or glioma.

In some embodiments of the methods disclosed herein, the hematologic cancer myeloid leukemia, acute lymphoblastic leukemia, marginal zone B cell lymphoma, acute lymphoblastic anemia, acute lymphocytic leukemia, lymphoma or thrombocythemia.

Chemotherapeutic agent: Any chemical agent with therapeutic usefulness in the treatment of diseases characterized by abnormal cell growth. Such diseases include tumors, neoplasms, and cancer as well as diseases characterized by hyperplastic growth such as psoriasis. One of skill in the art can readily identify a chemotherapeutic agent of use (see for example, Slapak and Kufe, Principles of Cancer Therapy, Chapter 86 in Harrison's Principles of Internal Medicine, 14th edition; Perry et al., Chemotherapy, Ch. 17 in Abeloff, Clinical Oncology 2^{nd} ed., © 2000 Churchill Livingstone, Inc.; Baltzer, L., Berkery, R. (eds.): Oncology Pocket Guide to Chemotherapy, 2nd ed. St. Louis, Mosby-Year Book, 1995; Fischer, D. S., Knobf, M. F., Durivage, H. J. (eds): The Cancer Chemotherapy Handbook, 4th ed. St. Louis, Mosby-Year Book, 1993). Combination chemotherapy is the administration of more than one agent to treat cancer. Chemotherapeutic agents are further described in section V below.

Choline-phosphate cytidylyltransferase alpha (CCTα): An enzyme involved in the biogenesis of phosphatidyl choline, a membrane component linked to cellular proliferation and Ras signaling activation in response to growth factor-induced mitogenic signaling. The CCTα protein is expressed in a number of different tissue types, including brain, lung, pancreas, intestine, uterus, testis, embryonic tissue, heart, kidney, skin, mouth, eye, blood, pharynx, larynx, prostate, mammary gland, muscle, adipose tissue, bone, liver, thymus, trachea, bladder, placenta, bone marrow, connective tissue, lymph node, spleen, adrenal gland, thyroid, ovary, esophagus, cervix, stomach, spinal cord, parathyroid, ascites, nerve, ganglia and amniotic fluid. The gene encoding CCTα is located at chromosome 3, location 3q29. Translocations at 3q29 have previously been associated with acute nonlymphocytic leukemia, lymphoma and thrombocythemia. The gene symbol for CCTα is PCYT1A and this gene is also known as CT, CTA, CCTA, CTPCT and PCYT1 (see NCBI database, gene ID 5130). Sequences for CCTα are publically available. For example, GenBank Accession Nos. NM_{—}005017 and NP_{—}005008 are nucleotide and amino acid sequences, respectively, of human CCTα.

Clinical outcome: Refers to the health status of a patient following treatment for a disease or disorder, or in the absence of treatment. Clinical outcomes include, but are not limited to, an increase in the length of time until death, a decrease in the length of time until death, an increase in the chance of survival, an increase in the risk of death, progression-free survival, survival, disease-free (or recurrence-free) survival, chronic disease, metastasis, advanced or aggressive disease, disease recurrence, death, tumor resistance or sensitivity to therapy, and favorable or poor response to therapy.

Control: A “control” refers to a sample or standard used for comparison with an experimental sample, such as a sample obtained from a cancer patient to be tested for expression of CCTα. In some embodiments, the control is a sample obtained from a healthy patient. In other embodiments, the control is a sample of normal tissue adjacent to tumor tissue obtained from the cancer patient. In yet other embodiments, the control is a historical control or reference standard (i.e. a previously tested control sample or group of samples that represent baseline or normal values, such as the level of CCTα expression in a healthy subject).

Detecting expression of a gene: Determining the existence, in either a qualitative or quantitative manner, of a particular nucleic acid or protein product (such as CCTα). Exemplary methods of detecting expression include RT-PCR, Northern blot, Western blot, immunohistochemistry, ELISA and mass spectrometry.

Genotoxic therapy: Any type of therapy that results in damage to DNA. Rapidly dividing cells (such as cancer cells) are particularly sensitive to genotoxic therapy because they are actively synthesizing DNA. Cells that are sufficiently damaged will undergo apoptosis (cell death). In the context of the present disclosure, genotoxic therapy includes radiation therapy and/or administration of a chemotherapeutic agent. Chemotherapeutic agents that are genotoxic include, for example, alkylating agents, crosslinking agents, intercalating agents and topoisomerase inhibitors.

Mutation: Any change of the DNA sequence within a gene or chromosome. In some instances, a mutation will alter a characteristic or trait (phenotype), but this is not always the case. Types of mutations include base substitution point mutations (e.g., transitions or transversions), deletions, insertions and chromosomal amplifications and translocations. Missense mutations are those that introduce a different amino acid into the sequence of the encoded protein; nonsense mutations are those that introduce a new stop codon. In the case of insertions or deletions, mutations can be in-frame (not changing the frame of the overall sequence) or frame shift mutations, which may result in the misreading of a large number of codons (and often leads to abnormal termination of the encoded product due to the presence of a stop codon in the alternative frame).

This term specifically encompasses variations that arise through somatic mutation, for instance those that are found only in disease cells, but not constitutionally, in a given individual. Examples of such somatically-acquired variations include the point mutations that frequently result in altered function of various genes that are involved in development of cancers. This term also encompasses DNA alterations that are present constitutionally, that alter the function of the encoded protein in a readily demonstrable manner, and that can be inherited by the children of an affected individual. In this respect, the term overlaps with “polymorphism,” but generally refers to the subset of constitutional alterations that have arisen within the past few generations in a kindred and that are not widely disseminated in a population group.

Prognosis: The likelihood of the clinical outcome for a subject afflicted with a specific disease or disorder. With regard to cancer, the prognosis is a representation of the likelihood (probability) that the subject will survive (such as for one, two, three, four or five years) and/or the likelihood (probability) that the tumor will metastasize, and/or the likelihood (probability) that the subject with survive without recurrence of the cancer. A “poor prognosis” indicates a greater than 50% chance that the subject will not survive to a specified time point (such as one, two, three, four or five years), and/or a greater than 50% chance that the tumor will metastasize. In several examples, a poor prognosis indicates that there is a greater than 60%, 70%, 80%, or 90% chance that the subject will not survive and/or a greater than 60%, 70%, 80% or 90% chance that the tumor will metastasize. Conversely, a “good prognosis” indicates a greater than 50% chance that the subject will survive to a specified time point (such as one, two, three, four or five years), and/or a greater than 50% chance that the tumor will not metastasize, and/or a greater than 50% chance that the subject with survive without recurrence of the cancer (referred to as “recurrence-free survival”). In several examples, a good prognosis indicates that there is a greater than 60%, 70%, 80%, or 90% chance that the subject will survive, and/or a greater than 60%, 70%, 80% or 90% chance that the tumor will not metastasize, and/or a greater than 60%, 70%, 80% or 90% chance that the subject with survive without recurrence of the cancer.

Sample or biological sample: As used herein, a “sample” obtained from a subject refers to a cell, fluid or tissue sample. Bodily fluids include, but are not limited to, blood, serum, urine and saliva. In some embodiments, the sample is a biopsy sample, such as needle aspiration biopsy.

Specifically detect: In the context of the present disclosure, “specifically detecting expression of CCTα” refers to detection of CCTα (using any means, such as Western blot, IHC, RT-PCR, Northern blot etc.) without detecting another marker, such as ERCC1.

Specific hybridization: Specific hybridization refers to the binding, duplexing, or hybridizing of a molecule only or substantially only to a particular nucleotide sequence when that sequence is present in a complex mixture (e.g. total cellular DNA or RNA). Specific hybridization may also occur under conditions of varying stringency.

Hybridization conditions resulting in particular degrees of stringency will vary depending upon the nature of the hybridization method of choice and the composition and length of the hybridizing DNA used. Generally, the temperature of hybridization and the ionic strength (especially the Na⁺ concentration) of the hybridization buffer will determine the stringency of hybridization. Calculations regarding hybridization conditions required for attaining particular degrees of stringency are discussed by Sambrook et al. (In: Molecular Cloning: A Laboratory Manual, Cold Spring Harbor, N.Y., 1989 ch. 9 and 11). By way of illustration only, a hybridization experiment may be performed by hybridization of a DNA molecule to a target DNA molecule which has been electrophoresed in an agarose gel and transferred to a nitrocellulose membrane by Southern blotting (Southern, J. Mol. Biol. 98:503, 1975), a technique well known in the art and described in Sambrook et al. (Molecular Cloning: A Laboratory Manual, Cold Spring Harbor, N.Y., 1989).

Traditional hybridization with a target nucleic acid molecule labeled with [³²P]-dCTP is generally carried out in a solution of high ionic strength such as 6×SSC at a temperature that is 20-25° C. below the melting temperature, Tₘ, described below. For Southern hybridization experiments where the target DNA molecule on the Southern blot contains 10 ng of DNA or more, hybridization is typically carried out for 6-8 hours using 1-2 ng/ml radiolabeled probe (of specific activity equal to 10⁹ CPM/μg or greater). Following hybridization, the nitrocellulose filter is washed to remove background hybridization. The washing conditions should be as stringent as possible to remove background hybridization but to retain a specific hybridization signal.

The term Tₘ represents the temperature (under defined ionic strength, pH and nucleic acid concentration) at which 50% of the probes complementary to the target sequence hybridize to the target sequence at equilibrium. Because the target sequences are generally present in excess, at Tₘ 50% of the probes are occupied at equilibrium. The Tₘ of such a hybrid molecule may be estimated from the following equation (Bolton and McCarthy, Proc. Natl. Acad. Sci. USA 48:1390, 1962):

Tₘ=81.5° C.−16.6(log₁₀[Na⁺])+0.41(% G+C)−0.63(% formamide)−(600/l)

where l=the length of the hybrid in base pairs.

This equation is valid for concentrations of Na⁺ in the range of 0.01 M to 0.4 M, and it is less accurate for calculations of Tm in solutions of higher [Na⁺]. The equation is also primarily valid for DNAs whose G+C content is in the range of 30% to 75%, and it applies to hybrids greater than 100 nucleotides in length (the behavior of oligonucleotide probes is described in detail in Ch. 11 of Sambrook et al. (Molecular Cloning: A Laboratory Manual, Cold Spring Harbor, N.Y., 1989).

Thus, by way of example, for a 150 base pair DNA probe derived from a cDNA (with a hypothetical % GC of 45%), a calculation of hybridization conditions required to give particular stringencies may be made as follows: For this example, it is assumed that the filter will be washed in 0.3×SSC solution following hybridization, thereby: [Na⁺]=0.045 M; % GC=45%; Formamide concentration=0; 1=150 base pairs; Tₘ=81.5−16.6(log₁₀[Na⁺])+(0.41×45)−(600/150); and so Tₘ=74.4° C.

The Tₘ of double-stranded DNA decreases by 1-1.5° C. with every 1% decrease in homology (Bonner et al., J. Mol. Biol. 81:123, 1973). Therefore, for this given example, washing the filter in 0.3×SSC at 59.4-64.4° C. will produce a stringency of hybridization equivalent to 90%; that is, DNA molecules with more than 10% sequence variation relative to the target cDNA will not hybridize. Alternatively, washing the hybridized filter in 0.3×SSC at a temperature of 65.4-68.4° C. will yield a hybridization stringency of 94%; that is, DNA molecules with more than 6% sequence variation relative to the target cDNA molecule will not hybridize. The above example is given entirely by way of theoretical illustration. It will be appreciated that other hybridization techniques may be utilized and that variations in experimental conditions will necessitate alternative calculations for stringency.

Stringent conditions may be defined as those under which DNA molecules with more than 25%, 15%, 10%, 6% or 2% sequence variation (also termed “mismatch”) will not hybridize. Stringent conditions are sequence dependent and are different in different circumstances. Longer sequences hybridize specifically at higher temperatures. Generally, stringent conditions are selected to be about 5° C. lower than the thermal melting point Tₘ for the specific sequence at a defined ionic strength and pH. An example of stringent conditions is a salt concentration of at least about 0.01 to 1.0 M Na ion concentration (or other salts) at pH 7.0 to 8.3 and a temperature of at least about 30° C. for short probes (e.g. 10 to 50 nucleotides). Stringent conditions can also be achieved with the addition of destabilizing agents such as formamide. For example, conditions of 5×SSPE (750 mM NaCl, 50 mM Na Phosphate, 5 mM EDTA, pH 7.4) and a temperature of 25-30° C. are suitable for allele-specific probe hybridizations.

The following is an exemplary set of hybridization conditions and is not meant to be limiting:

Very High Stringency (Detects Sequences that Share 90% Identity)

Hybridization: 5×SSC at 65° C. for 16 hours

Wash twice: 2×SSC at room temperature (RT) for 15 minutes each

Wash twice: 0.5×SSC at 65° C. for 20 minutes each

High Stringency (Detects Sequences that Share 80% Identity or Greater)

Hybridization: 5×-6×SSC at 65° C.-70° C. for 16-20 hours

Wash twice: 2×SSC at RT for 5-20 minutes each

Wash twice: 1×SSC at 55° C.-70° C. for 30 minutes each

Low Stringency (Detects Sequences that Share Greater than 50% Identity)

Hybridization: 6×SSC at RT to 55° C. for 16-20 hours

Wash at least twice: 2×-3×SSC at RT to 55° C. for 20-30 minutes each.

A perfectly matched probe has a sequence perfectly complementary to a particular target sequence. The test probe is typically perfectly complementary to a portion (subsequence) of the target sequence. The term “mismatch probe” refers to probes whose sequence is deliberately selected not to be perfectly complementary to a particular target sequence.

Subject: As used herein, the term “subject” includes human and non-human animals. The preferred subject for prognosis and/or treatment is a human. “Patient” and “subject” are used interchangeably herein.

Unless otherwise explained, all technical and scientific terms used herein have the same meaning as commonly understood by one of ordinary skill in the art to which this disclosure belongs. The singular terms “a,” “an,” and “the” include plural referents unless context clearly indicates otherwise. Similarly, the word “or” is intended to include “and” unless the context clearly indicates otherwise. Hence “comprising A or B” means including A, or B, or A and B. It is further to be understood that all base sizes or amino acid sizes, and all molecular weight or molecular mass values, given for nucleic acids or polypeptides are approximate, and are provided for description. Although methods and materials similar or equivalent to those described herein can be used in the practice or testing of the present disclosure, suitable methods and materials are described below. All publications, patent applications, patents, and other references mentioned herein are incorporated by reference in their entirety. All GenBank Accession numbers are herein incorporated by reference as they appear in the database on Feb. 1, 2011. In case of conflict, the present specification, including explanations of terms, will control. In addition, the materials, methods, and examples are illustrative only and not intended to be limiting.

### III. Introduction

Platinum-based chemotherapy is the cornerstone of therapy for NSCLC and HNSCC, but is plagued with a high frequency of drug resistance and toxic side effects. The identification of molecular markers to predict therapeutic efficacy is a prerequisite for personalized cancer therapy and improving poor outcomes. DNA repair proteins are good biomarker candidates. They are involved in maintaining genetic stability, implicated in cancer initiation, progression and response to genotoxic therapeutic agents. ERCC1-XPF is a bipartite endonuclease essential for nucleotide excision repair of bulky DNA adducts and interstrand crosslink repair, the types of damage caused by platinum agents (Niedernhofer et al., Nature 444(7122): 1038-1043, 2006; Ahmad et al., Mol Cell Biol 28(16): 5082-5092, 2008; Bhagwat et al., Mol Cell Biol 29(24): 6427-6437, 2009; Vaezi et al., Clin Cancer Res 17(16): 5513-5522, 2011). In patients with early-stage NSCLC treated with surgery alone, high levels of ERCC1 protein are associated with longer survival, presumably a result of maintaining genome stability (Olaussen et al., N Engl J Med 355(10): 983-991, 2006; Zheng et al., N Engl J Med 356(8): 800-808, 2007). However, in patients treated with genotoxic agents, low ERCC1 expression is associated with better survival (Olaussen et al., N Engl J Med 355(10): 983-991, 2006), presumably because of greater drug sensitivity as a result of reduced repair capacity. The relationship between ERCC1 and survival in NSCLC treated with DNA damaging agents was confirmed in Phase III clinical trial (Reynolds et al., J Clin Oncol 27(34): 5808-5815, 2009) and meta-analyses (Jiang et al., Mol Biol Rep 39(6): 6933-6942, 2012; Roth and Carlson, Clin Lung Cancer 12(6): 393-401, 2011; Chen et al., Lung Cancer 70(1): 63-70, 2010). ERCC1 is currently used as an enrichment biomarker in clinical trials. However, the studies that established the predictive value of ERCC1 protein expression as a biomarker invariably used the monoclonal antibody 8F1.

It has been previously demonstrated that 8F1 lacks specificity for ERCC1 (Niedernhofer et al., N Engl J Med 356(24): 2538-2540; author reply 40-1, 2007). In addition to reacting with ERCC1, 8F1 recognizes a second nuclear protein in immunoblots and IHC (Niedernhofer et al., N Engl J Med 356(24): 2538-2540; author reply 40-1, 2007). This cross-reactivity was important, as 8F1 was unable to discriminate between normal and ERCC1-XPF-deficient cells (Bhagwat et al., Cancer Res 69(17): 6831-6838, 2009). It is therefore possible that studies using 8F1 measured signals corresponding to both proteins. Notably, in lung cancer, an XPF-specific antibody does not predict outcome, whereas 8F1 does (Planchard et al., Ann Oncol 20(7): 1257-1263, 2009; Pierceall et al., Ann Oncol 23(9): 2245-2252, 2012). In HNSCC, a specific ERCC1 antibody (FL297) predicted survival, while 8F1 did not (Hao et al., Head Neck 34(6): 785-91, 2012).

As disclosed herein, using mass spectrometry, the second antigen recognized by 8F1 was identified as choline-phosphate cytidylyltransferase-α (CCTα), a rate-limiting enzyme involved in the synthesis of phosphatidyl choline. It was confirmed that CCTα is recognized by 8F1, and that this cross-reactivity significantly influences 8F1 signal by IHC in clinical samples. It is further disclosed herein that CCTα protein expression is a determinant of clinical outcomes in two independent cohorts of squamous cell carcinomas. Thus, CCTα, the second protein identified by 8F1, possesses intrinsic biomarker value for the prognosis of cancer.

### IV. Overview of Several Embodiments

In oncology, it is imperative to discover prognostic biomarkers (instructive on the natural course of the disease) and predictive biomarkers (instructive on outcome after a given treatment) to identify patients at risk of recurrence and therefore poor clinical outcomes. It has previously been demonstrated that positive 8F1 immunohistochemistry is predictive of a patient's response to chemoradiation therapy and therefore patient outcome. It has also previously been demonstrated that positive 8F1 immunohistochemistry is prognostic for cancer patient outcomes. The 8F1 antibody was generated by immunization against ERCC1, a DNA repair protein. However, it has previously been reported that the 8F1 antibody recognizes a second unidentified protein in cells and tissues (Bhagwat et al., Cancer Res 69:6831-6838, 2009; Niedernhofer et al., N Eng J. Med. 356:2538-2541, 2007). Disclosed herein is the finding that in addition to ERCC1, the 8F1 antibody recognizes the choline phosphate cytidylyltransferase-α (CCTα) protein and that a high level of CCTα expression in patient samples is associated with longer, recurrence-free survival.

Thus, provided herein is a method of determining the prognosis of a patient with cancer. In some embodiments, the method includes specifically detecting expression of CCTα in a sample obtained from the subject. An increase in expression of CCTα in the sample compared to a control indicates a good prognosis for the patient. In some embodiments, specifically detecting expression of CCTα in a sample comprises detecting expression of CCTα without detecting expression of ERCC1 in the sample.

Also provided is a method of predicting the response of a cancer patient to treatment with a genotoxic therapy. In some embodiments, the method includes specifically detecting expression of CCTα in a sample obtained from the subject. An increase in expression of CCTα in the sample compared to a control predicts a poor response to the genotoxic agent. In some embodiments, specifically detecting expression of CCTα in a sample comprises detecting expression of CCTα without detecting expression of ERCC1 in the sample.

Detecting expression of CCTα can include detecting CCTα protein, detecting CCTα mRNA, or both. In alternative embodiments of the methods, the identification of an amplification, translocation or other mutation in the chromosomal locus encoding CCTα (3q29) indicates a good prognosis and/or poor response to genotoxic therapy.

The relative change in expression level of CCTα that is indicative of a good prognosis or poor response to genotoxic therapy can vary and will differ depending on whether one is detecting expression of CCTα protein or CCTα mRNA. In some embodiments, expression of CCTα protein is increased at least 2-fold, at least 3-fold, at least 4-fold or at least 5-fold relative to the control; expression of CCTα mRNA is increased at least 5-fold, at least 10-fold, at least 25-fold or at least 50-fold relative to the control; or both. In some embodiments, expression of CCTα protein and/or CCTα mRNA is increased at least 10%, at least 15%, at least 20%, at least 25%, at least 30%, at least 40%, at least 50%, at least 60%, at least 70% or at least 80%.

Detection of CCTα protein or CCTα mRNA can be achieved using any suitable method known in the art. Exemplary methods of mRNA and protein detection are described in greater detail in section V. In some embodiments, CCTα mRNA is detected by RT-PCR, Northern blot or in situ hybridization. In some embodiments, CCTα protein is detected by IHC, immunofluorescence, Western blot or ELISA. The method of detection will depend in part on the type of sample to be tested.

The sample obtained from the subject can be any suitable sample for detection of CCTα protein or CCTα mRNA. In some embodiments, the sample is a cell or tissue sample, such as a tumor sample. In other embodiments, the sample is a bodily fluid sample, such as a blood sample.

The control used in the disclosed methods can be any control sample or value that represents the level of CCTα expression in a healthy individual. In some embodiments, the control is a non-tumor sample obtained from the subject or a sample from a healthy subject. In other embodiments, the control is a reference value.

The disclosed methods can be used to evaluate the prognosis of a patient (or response to chemotherapy of a patient) with any type of cancer, including patients with solid tumors and patients with hematologic cancers.

In some embodiments, the cancer is a solid tumor. In particular examples, the solid tumor is non-small cell lung carcinoma, gastric carcinoma, esophageal carcinoma, pancreatic cancer, colon cancer, breast cancer, brain cancer, head and neck squamous cell carcinoma, squamous cell carcinoma of the lung, pulmonary papillary adenocarcinoma, mesothelioma, esophageal cancer, nasopharyngeal cancer, prostate cancer, adrenocortical carcinoma, cutaneous neuroendocrine cancer, gallbladder cancer, bile duct cancer, cervical cancer, serous ovarian cancer, epithelial ovarian cancer, endometrial cancer, bladder cancer, urothelial carcinoma, Ewing sarcoma, testicular cancer, neuroendocrine cancer, liver cancer, hepatocellular carcinoma, pituitary cancer or glioma.

In other embodiments, the cancer is a hematologic cancer, such as a leukemia or a lymphoma. In particular non-limiting examples, the hematologic cancer is myeloid leukemia, acute lymphoblastic leukemia, marginal zone B cell lymphoma, acute lymphoblastic anemia, acute lymphocytic leukemia, lymphoma or thrombocythemia.

In some embodiments, the cancer is a cancer of the upper aerodigestive tract, such as, but not limited to head and neck squamous cell carcinoma (HNSCC), oral carcinoma, gastric carcinoma, esophageal carcinoma or nasopharyngeal cancer. Other aerodigestive tract cancers include, for example, cancers of the lip, tongue, major salivary glands, gums and adjacent oral cavity tissues, floor of the mouth, tonsils, oropharynx, nasopharynx, hypopharynx and other oral regions, such as the nasal cavity, accessory sinuses, middle ear, and larynx (Muir and Weiland, Cancer 75(Suppl):147-153, 1995).

In some embodiments, the cancer is a squamous cell carcinoma, such as head and neck squamous cell carcinoma or squamous cell carcinoma of the lung.

In some embodiments, the cancer comprises a tumor harboring a Ras mutation. In some instances, the cancer with a ras mutation is pancreatic cancer or colon cancer.

The genotoxic therapy can be any type of therapy that induces DNA damage (see section VI below). In some embodiments, the genotoxic therapy comprises radiation therapy. In other embodiments, the genotoxic therapy comprises administration of a genotoxic agent (e.g., a drug), such as a chemotherapeutic agent. In some examples, the chemotherapeutic agent is a platinum-based chemotherapeutic agent, such as, but not limited to cisplatin, carboplatin or oxaliplatin. In other examples, the chemotherapeutic agent is an alkylating agent, such as, but not limited to BCNU, cyclophosphamide, melphalan, mitomycin C, mechlorethamine or a psoralen. In yet other examples, the chemotherapeutic agent is bleomycin, doxorubicin or etoposide.

In some embodiments, the method further includes providing a test output (i.e., the result of the test to detect expression of CCTα in a sample) to a user (such as a physician or health care worker, the patient or laboratory personnel). In particular examples, the output includes the presence or absence of expression and/or relative expression level of CCTα, a diagnosis, a treatment recommendation, or any combination thereof. Examples of such output include a printout or display screen that reports the output by displaying it to a clinician or technician. Other examples are electronic medical record reports or other records that include the output in a form discernible to the clinician or technician.

Further provided herein is a method of determining the prognosis of a patient with cancer by detecting an amplification, translocation or other mutation in the chromosomal locus (3q29) of CCTα in a sample obtained from the subject. The presence of an amplification, translocation or other mutation that increases expression of CCTα indicates a good prognosis for the patient. Also provided is a method of predicting the response of a cancer patient to treatment with a genotoxic therapy by detecting an amplification, translocation or other mutation in the chromosomal locus (3q29) of CCTα in a sample obtained from the subject. The presence of an amplification, translocation or other mutation that increases expression of CCTα predicts a poor response to the genotoxic agent.

In some embodiments, the method does not detect expression of ERCC1.

In some embodiments, the methods disclosed herein further include selecting and/or administering an appropriate therapy to the patient with cancer. In some examples, the appropriate therapy comprises genotoxic therapy if a decrease in expression of CCTα is detected in the sample compared to a control. In some examples, the appropriate therapy further comprises administration of an agent that inhibits expression of CCTα.

Also provided herein is a method of improving the clinical outcome of a patient with cancer, wherein the patient has undergone or will undergo genotoxic therapy, comprising administering to the patient an agent that inhibits expression of CCTα. Inhibitors can include, for example, small molecule inhibitors or antisense compounds specific for CCTα (e.g., antisense oligonucleotides, siRNAs, shRNAs and the like).

Further provided is a method of predicting a subject's risk of developing cancer, comprising detecting expression of CCTα in a sample obtained from the subject. An increase in expression of CCTα relative to a control indicates that the subject has a relatively low risk of cancer (e.g. compared to the general population). A decrease in expression of CCTα relative to a control indicates the subject has a relatively high risk of cancer (e.g. compared to the general population). In some embodiments, the control is a historical control or reference standard (i.e. a previously tested control sample or group of samples that represent baseline or normal values, such as the level of CCTα expression in a healthy subject).

### V. Detecting Expression of CCTα

As described below, expression of CCTα can be detected using any one of a number of methods well known in the art. Expression of either mRNA or protein is contemplated herein.

A. Methods for Detection of CCTα mRNA

Gene expression can be evaluated by detecting mRNA of the gene of interest. Thus, the disclosed methods can include evaluating CCTα mRNA. In some examples, the mRNA is quantified.

RNA can be isolated from a sample using methods well known to one skilled in the art, including commercially available kits. The sample can be obtained from a subject with cancer and/or a control subject. The sample can be any suitable biological sample, such as a bodily fluid sample (such as blood) or a tissue sample (such as a tumor tissue sample).

General methods for mRNA extraction are well known in the art and are disclosed in standard textbooks of molecular biology, including Ausubel et al., Current Protocols of Molecular Biology, John Wiley and Sons (1997). Methods for RNA extraction from paraffin embedded tissues are disclosed, for example, in Rupp and Locker (Lab Invest. 56:A67, 1987), and De Andres et al. (BioTechniques 18:42044, 1995). In some examples, RNA isolation can be performed using a purification kit, buffer set and protease from commercial manufacturers, such as QIAGEN™, according to the manufacturer's instructions. For example, total RNA from cells in a sample (such as those obtained from a subject) can be isolated using QIAGIN™ RNeasy mini-columns. Other commercially available RNA isolation kits include MASTERPURE™. Complete DNA and RNA Purification Kit (EPICENTRE™ Madison, Wis.), and Paraffin Block RNA Isolation Kit (Ambion, Inc.). Total RNA from tissue samples can be isolated, for example, using RNA Stat-60 (Tel-Test). RNA prepared from a biological sample can be isolated, for example, by cesium chloride density gradient centrifugation.

Methods of gene expression profiling include methods based on hybridization analysis of polynucleotides, methods based on sequencing of polynucleotides, and proteomics-based methods. In some examples, mRNA expression in a sample is quantified using northern blotting or in situ hybridization (Parker & Barnes, Methods in Molecular Biology 106:247-283, 1999); RNAse protection assays (Hod, Biotechniques 13:852-854, 1992); and PCR-based methods, such as reverse transcription polymerase chain reaction (RT-PCR) (Weis et al., Trends in Genetics 8:263-264, 1992). Representative methods for sequencing-based gene expression analysis include Serial Analysis of Gene Expression (SAGE), and gene expression analysis by massively parallel signature sequencing (MPSS). In one example, RT-PCR can be used to compare mRNA levels in different samples (such as samples from cancer subjects and healthy subjects) to characterize patterns of gene expression.

Methods for quantifying mRNA are well known in the art. In some examples, the method utilizes RT-PCR. Generally, the first step in gene expression profiling by RT-PCR is the reverse transcription of the RNA template into cDNA, followed by its exponential amplification in a PCR reaction. Two commonly used reverse transcriptases are avian myeloblastosis virus reverse transcriptase (AMV-RT) and Moloney murine leukemia virus reverse transcriptase (MMLV-RT). The reverse transcription step is typically primed using specific primers, random hexamers, or oligo-dT primers, depending on the circumstances and the goal of expression profiling. For example, extracted RNA can be reverse-transcribed using a GeneAmp™ RNA PCR kit (Perkin Elmer, Calif.), following the manufacturer's instructions. The derived cDNA can then be used as a template in the subsequent PCR reaction.

Although the PCR step can use a variety of thermostable DNA-dependent DNA polymerases, it typically employs the Taq DNA polymerase, which has a 5′-3′ nuclease activity but lacks a 3′-5′ proofreading endonuclease activity. TaqMan™ PCR typically utilizes the 5′-nuclease activity of Taq or Tth polymerase to hydrolyze a hybridization probe bound to its target amplicon, but any enzyme with equivalent 5′ nuclease activity can be used. Two oligonucleotide primers are used to generate an amplicon typical of a PCR reaction. A third oligonucleotide, or probe, is designed to detect nucleotide sequence located between the two PCR primers. The probe is non-extendible by Taq DNA polymerase enzyme, and is labeled with a reporter fluorescent dye and a quencher fluorescent dye. Any laser-induced emission from the reporter dye is quenched by the quenching dye when the two dyes are located close together as they are on the probe. During the amplification reaction, the Taq DNA polymerase enzyme cleaves the probe in a template-dependent manner. The resultant probe fragments disassociate in solution, and signal from the released reporter dye is free from the quenching effect of the second fluorophore. One molecule of reporter dye is liberated for each new molecule synthesized, and detection of the unquenched reporter dye provides the basis for quantitative interpretation of the data.

TAQMAN™ RT-PCR can be performed using commercially available equipment, such as, for example, ABI PRISM 7700™ Sequence Detection System™ (Perkin-Elmer-Applied Biosystems, Foster City, Calif.), or Lightcycler (Roche Molecular Biochemicals, Mannheim, Germany). In one example, the 5′ nuclease procedure is run on a real-time quantitative PCR device such as the ABI PRISM 7700™ Sequence Detection System™. The system includes of thermocycler, laser, charge-coupled device (CCD), camera and computer. The system amplifies samples in a 96-well format on a thermocycler. During amplification, laser-induced fluorescent signal is collected in real-time through fiber optics cables for all 96 wells, and detected at the CCD. The system includes software for running the instrument and for analyzing the data.

To minimize errors and the effect of sample-to-sample variation, RT-PCR can be performed using an internal standard. The ideal internal standard is expressed at a constant level among different tissues, and is unaffected by the experimental treatment. RNAs commonly used to normalize patterns of gene expression are mRNAs for the housekeeping genes glyceraldehyde-3-phosphate-dehydrogenase (GAPDH), beta-actin, and 18S ribosomal RNA.

A variation of RT-PCR is real time quantitative RT-PCR, which measures PCR product accumulation through a dual-labeled fluorogenic probe (e.g., TAQMAN™ probe). Real time PCR is compatible both with quantitative competitive PCR, where internal competitor for each target sequence is used for normalization, and with quantitative comparative PCR using a normalization gene contained within the sample, or a housekeeping gene for RT-PCR (see Held et al., Genome Research 6:986-994, 1996). Quantitative PCR is also described in U.S. Pat. No. 5,538,848. Related probes and quantitative amplification procedures are described in U.S. Pat. No. 5,716,784 and U.S. Pat. No. 5,723,591. Instruments for carrying out quantitative PCR in microtiter plates are available, for example, from PE Applied Biosystems, under the trademark ABI PRISM™ 7700.

The steps of a representative protocol for quantifying gene expression using fixed, paraffin-embedded tissues as the RNA source, including mRNA isolation, purification, primer extension and amplification are given in various publications (see Godfrey et al., J. Mol. Diag. 2:84-91, 2000; Specht et al., Am. J. Pathol. 158:419-429, 2001). Briefly, a representative process starts with cutting about 10 μm thick sections of paraffin-embedded tumor tissue samples or adjacent non-cancerous tissue. The RNA is then extracted, and protein and DNA are removed. Alternatively, RNA is located directly from a tumor sample or other tissue sample. After analysis of the RNA concentration, RNA repair and/or amplification steps can be included, if necessary, and RNA is reverse transcribed using gene specific promoters followed by RT-PCR. The primers used for the amplification are selected so as to amplify a unique segment of the gene of interest, such as CCTα mRNA. Primers that can be used to amplify CCTα are commercially available or can be designed and synthesized according to well known methods.

An alternative quantitative nucleic acid amplification procedure is described in U.S. Pat. No. 5,219,727. In this procedure, the amount of a target sequence in a sample is determined by simultaneously amplifying the target sequence and an internal standard nucleic acid segment. The amount of amplified DNA from each segment is determined and compared to a standard curve to determine the amount of the target nucleic acid segment that was present in the sample prior to amplification.

In some embodiments of this method, the expression of a “housekeeping” gene or “internal control” can also be evaluated. These terms include any constitutively or globally expressed gene whose presence enables an assessment of CCTα mRNA levels. Such an assessment includes a determination of the overall constitutive level of gene transcription and a control for variations in RNA recovery.

In some examples, gene expression is identified or confirmed using the microarray technique. In this method, CCTα nucleic acid sequences of interest (including cDNAs and oligonucleotides) are plated, or arrayed, on a microchip substrate. The arrayed sequences are then hybridized with specific DNA probes from cells or tissues of interest.

In a specific embodiment of the microarray technique, PCR amplified inserts of cDNA clones are applied to a substrate in a dense array. At least probes specific for CCTα nucleotide sequences are applied to the substrate. The microarrayed nucleic acids are suitable for hybridization under stringent conditions. Fluorescently labeled cDNA probes may be generated through incorporation of fluorescent nucleotides by reverse transcription of RNA extracted from tissues of interest. Labeled cDNA probes applied to the chip hybridize with specificity to each spot of DNA on the array. After stringent washing to remove non-specifically bound probes, the chip is scanned by confocal laser microscopy or by another detection method, such as a CCD camera. Quantitation of hybridization of each arrayed element allows for assessment of corresponding mRNA abundance. With dual color fluorescence, separately labeled cDNA probes generated from two sources of RNA are hybridized pairwise to the array. The relative abundance of the transcripts from the two sources corresponding to each specified gene is thus determined simultaneously.

Serial analysis of gene expression (SAGE) is another method that allows the simultaneous and quantitative analysis of a large number of gene transcripts, without the need of providing an individual hybridization probe for each transcript. First, a short sequence tag (about 10-14 base pairs) is generated that contains sufficient information to uniquely identify a transcript, provided that the tag is obtained from a unique position within each transcript. Then, many transcripts are linked together to form long serial molecules, that can be sequenced, revealing the identity of the multiple tags simultaneously. The expression pattern of any population of transcripts can be quantitatively evaluated by determining the abundance of individual tags, and identifying the gene corresponding to each tag (see, for example, Velculescu et al., Science 270:484-7, 1995; and Velculescu et al., Cell 88:243-51, 1997).

In situ hybridization (ISH) is another method for detecting and comparing expression of genes of interest. ISH applies and extrapolates the technology of nucleic acid hybridization to the single cell level, and, in combination with the art of cytochemistry, immunocytochemistry and immunohistochemistry, permits the maintenance of morphology and the identification of cellular markers to be maintained and identified, and allows the localization of sequences to specific cells within populations, such as tissues and blood samples. ISH is a type of hybridization that uses a complementary nucleic acid to localize one or more specific nucleic acid sequences in a portion or section of tissue (in situ), or, if the tissue is small enough, in the entire tissue (whole mount ISH). RNA ISH can be used to assay expression patterns in a tissue, such as the expression of CCTα.

Sample cells or tissues are treated to increase their permeability to allow a probe, such as a CCTα-specific probe, to enter the cells. The probe is added to the treated cells, allowed to hybridize at pertinent temperature, and excess probe is washed away. A complementary probe is labeled with a radioactive, fluorescent or antigenic tag, so that the probe's location and quantity in the tissue can be determined using autoradiography, fluorescence microscopy or immunoassay. The sample may be any sample as herein described, such as a blood sample or tissue sample obtained from a subject with cancer. Since the sequence of CCTα is known, probes can be designed accordingly such that the probes specifically bind CCTα.

In situ PCR is the PCR based amplification of the target nucleic acid sequences prior to ISH. For detection of RNA, an intracellular reverse transcription step is introduced to generate complementary DNA from RNA templates prior to in situ PCR. This enables detection of low copy RNA sequences.

Prior to in situ PCR, cells or tissue samples are fixed and permeabilized to preserve morphology and permit access of the PCR reagents to the intracellular sequences to be amplified. PCR amplification of target sequences is next performed either in intact cells held in suspension or directly in cytocentrifuge preparations or tissue sections on glass slides. In the former approach, fixed cells suspended in the PCR reaction mixture are thermally cycled using conventional thermal cyclers. After PCR, the cells are cytocentrifuged onto glass slides with visualization of intracellular PCR products by ISH or immunohistochemistry. In situ PCR on glass slides is performed by overlaying the samples with the PCR mixture under a coverslip which is then sealed to prevent evaporation of the reaction mixture. Thermal cycling is achieved by placing the glass slides either directly on top of the heating block of a conventional or specially designed thermal cycler or by using thermal cycling ovens.

Detection of intracellular PCR products is generally achieved by one of two different techniques, indirect in situ PCR by ISH with PCR-product specific probes, or direct in situ PCR without ISH through direct detection of labeled nucleotides (such as digoxigenin-11-dUTP, fluorescein-dUTP, 3H-CTP or biotin-16-dUTP), which have been incorporated into the PCR products during thermal cycling.

B. Methods for Detection of CCTα Protein

In some examples, expression of CCTα protein is analyzed. Antibodies specific for CCTα protein can be used for detection and quantification of CCTα by one of a number of immunoassay methods that are well known in the art, such as those presented in Harlow and Lane (Antibodies, A Laboratory Manual, CSHL, New York, 1988). Methods of constructing such antibodies are known in the art.

Any standard immunoassay format (such as ELISA, Western blot, or RIA assay) can be used to measure protein levels. Thus, CCTα polypeptide levels in a sample (such as a blood sample or tissue sample) can readily be evaluated using these methods. Immunohistochemical techniques can also be utilized for CCTα detection and quantification. General guidance regarding such techniques can be found in Bancroft and Stevens (Theory and Practice of Histological Techniques, Churchill Livingstone, 1982) and Ausubel et al. (Current Protocols in Molecular Biology, John Wiley & Sons, New York, 1998).

For the purposes of quantifying CCTα, a biological sample of the subject that includes cellular proteins can be used. Quantification of CCTα protein can be achieved by immunoassay methods known in the art. The amount CCTα protein can be assessed in samples from cancer patients and/or in samples from cancer-free subjects. The amounts of CCTα protein in the sample can be compared to a control, such as the levels of the proteins found in cells from a cancer-free subject or other control (such as a standard value or reference value). A significant increase or decrease in the amount can be evaluated using statistical methods disclosed herein and/or known in the art.

Quantitative spectroscopic approaches methods, such as SELDI, can be used to analyze CCTα expression in a sample. In one example, surface-enhanced laser desorption-ionization time-of-flight (SELDI-TOF) mass spectrometry is used to detect protein expression, for example by using the ProteinChip™ (Ciphergen Biosystems, Palo Alto, Calif.). Such methods are well known in the art (for example see U.S. Pat. Nos. 5,719,060; 6,897,072; and 6,881,586). SELDI is a solid phase method for desorption in which the analyte is presented to the energy stream on a surface that enhances analyte capture or desorption.

Briefly, one version of SELDI uses a chromatographic surface with a chemistry that selectively captures analytes of interest, such as CRF-associated proteins. Chromatographic surfaces can be composed of hydrophobic, hydrophilic, ion exchange, immobilized metal, or other chemistries. For example, the surface chemistry can include binding functionalities based on oxygen-dependent, carbon-dependent, sulfur-dependent, and/or nitrogen-dependent means of covalent or noncovalent immobilization of analytes. The activated surfaces are used to covalently immobilize specific “bait” molecules such as antibodies, receptors, or oligonucleotides often used for biomolecular interaction studies such as protein-protein and protein-DNA interactions.

The surface chemistry allows the bound analytes to be retained and unbound materials to be washed away. Subsequently, analytes bound to the surface (such as CRF-associated proteins) can be desorbed and analyzed by any of several means, for example using mass spectrometry. When the analyte is ionized in the process of desorption, such as in laser desorption/ionization mass spectrometry, the detector can be an ion detector. Mass spectrometers generally include means for determining the time-of-flight of desorbed ions. This information is converted to mass. However, one need not determine the mass of desorbed ions to resolve and detect them: the fact that ionized analytes strike the detector at different times provides detection and resolution of them. Alternatively, the analyte can be detectably labeled (for example with a fluorophore or radioactive isotope). In these cases, the detector can be a fluorescence or radioactivity detector. A plurality of detection means can be implemented in series to fully interrogate the analyte components and function associated with retained molecules at each location in the array.

Therefore, in a particular example, the chromatographic surface includes antibodies that specifically bind CCTα. In some examples, the chromatographic surface includes antibodies that bind other molecules, such as housekeeping proteins (e.g. actin or myosin).

In another example, antibodies are immobilized onto the surface using a bacterial Fc binding support. The chromatographic surface is incubated with a sample, such as a blood sample or tissue sample (such as a tumor biopsy). The antigens present in the sample can recognize the antibodies on the chromatographic surface. The unbound proteins and mass spectrometric interfering compounds are washed away and the proteins that are retained on the chromatographic surface are analyzed and detected by SELDI-TOF. The MS profile from the sample can be then compared using differential protein expression mapping, whereby relative expression levels of proteins at specific molecular weights are compared by a variety of statistical techniques and bioinformatic software systems.

C. Antibodies for Detection of CCTα Protein

As discussed above, expression of CCTα protein in a sample can be detected using any one of a number of techniques, including immunological detection methods using antibodies specific for CCTα. A number of antibodies that were raised against CCTα protein are commercially available (see Table 1 below) and can be used in a number of different immunological-based applications, including Western blot (WB), flow cytometry (Flow Cyt), immunohistochemistry (IHC), immunoprecipitation (IP), enzyme-linked immunosorbent assay (ELISA), immunocytochemistry (ICC), immunofluorescence (IF), and protein array.

TABLE 1
Commercial antibodies for detection of CCTα

| | | | | | Applications |
|---|---|---|---|---|---|
| Name | Source | mono/poly | epitope | Origin | listed |
| EPR3941 | GeneTex | mono | full length | rabbit | Flow Cyt, |
| | | | human | | ICC/IF, IP, |
| | | | | | WB |
| Clone | Abcam | mono | full length | rabbit | Flow Cyt, |
| EPR3941 | | | human | | ICC/IF, IP, |
| (ab92440) | | | | | WB |
| Clone | LifeSpan | mono | full length | rabbit | Flow Cyt, |
| EPR3941 | Biosciences | | human | | ICC/IF, IP, |
| | | | | | WB |
| EPR3941 | Novus | mono | full length | rabbit | Flow Cyt, |
| | Biologicals | | human | | ICC/IF, IP, |
| | | | | | WB |
| H00005130- | Novus | poly | full length | rabbit | WB, ELISA |
| D01P | Biologicals | | human | | |
| H00005130- | Abnova | poly | full length | rabbit | WB |
| D01P | | | human | | |
| H00005130- | Abnova | poly | amino acids 2- | mouse | ELISA, WB |
| A01 | | | 91 of Human | | |
| | | | PCYT1A | | |
| H00005130- | Novus | poly | amino acids 2- | mouse | ELISA, WB |
| A01 | Biologicals | | 91 of Human | | |
| | | | PCYT1A | | |
| HPA035428 | Atlas | poly | amino acids 28- | rabbit | IHC, WB, |
| | Antibodies | | 78 of Human | | Protein Array |
| | | | PCYT1A | | |
| HPA035428 | Sigma | poly | amino acids 28- | rabbit | IHC, WB, |
| | Aldrich | | 78 of Human | | Protein Array |
| | | | PCYT1A | | |
| Clone 7H8 | Novus | mono | amino acids 2- | mouse | ELISA, WB |
| | Biologicals | | 91 of Human | | |
| | | | PCYT1A | | |
| Clone 7H8 | Abgent | mono | amino acids 2- | Mouse | ELISA, WB, |
| | | | 91 of Human | | sELISA |
| | | | PCYT1A | | |
| Clone 7H8 | Abnova | mono | amino acids 2- | Mouse | ELISA, WB, |
| | | | 91 of Human | | sELISA |
| | | | PCYT1A | | |
| Clone 7H8 | Sigma | mono | amino acids 2- | Mouse | indirect |
| | Aldrich | | 91 of Human | | ELISA, WB |
| | | | PCYT1A | | |
| Clone 6E6 | AbD | mono | amino acids 2- | Mouse | WB |
| | Serotec | | 91 of Human | | |
| | | | PCYT1A | | |
| Clone 6E6 | Abgent | mono | amino acids 2- | Mouse | ELISA, WB |
| | | | 91 of Human | | |
| | | | PCYT1A | | |
| Clone 6E6 | Abnova | mono | amino acids 2- | Mouse | ELISA, WB, |
| | | | 91 of Human | | sELISA |
| | | | PCYT1A | | |
| Clone 6E6 | Novus | mono | amino acids 2- | mouse | ELISA, WB |
| | Biologicals | | 91 of Human | | |
| | | | PCYT1A | | |
| F-17 | Santa Cruz | poly | peptide mapping | goat | WB, solid |
| | Biotech | | to an internal | | phase |
| | | | region of human | | ELISA, IF |
| | | | CCTα | | |
| N-20 | Santa Cruz | poly | N-terminal | goat | WB, IP, IF, |
| | Biotech | | peptide of | | solid phase |
| | | | human CCTA | | ELISA |
| W-13 | Santa Cruz | mono | full length | mouse | WB, IP, solid |
| | Biotech | | human | | phase ELISA |
| SAB1401279 | Sigma | poly | full length | rabbit | WB |
| | Aldrich | | human | | |
| ab77305 | Abcam | mono | amino acids 2- | mouse | ELISA, WB, |
| | | | 91 of Human | | sELISA |
| | | | PCYT1A | | |
| ab55554 | Abcam | mono | amino acids 2- | mouse | WB |
| | | | 91 of Human | | |
| | | | PCYT1A | | |

Moreover, Methods of making polyclonal and monoclonal antibodies are well known in the art. Polyclonal antibodies, antibodies which consist essentially of pooled monoclonal antibodies with different epitopic specificities, as well as distinct monoclonal antibody preparations are included. The preparation of polyclonal antibodies is well known to those skilled in the art (see, for example, Green et al., “Production of Polyclonal Antisera,” in: Immunochemical Protocols, pages 1-5, Manson, ed., Humana Press, 1992; Coligan et al., “Production of Polyclonal Antisera in Rabbits, Rats, Mice and Hamsters,” in: Current Protocols in Immunology, section 2.4.1, 1992).

The preparation of monoclonal antibodies likewise is conventional (see, for example, Kohler & Milstein, Nature 256:495, 1975; Coligan et al., sections 2.5.1-2.6.7; and Harlow et al. in: Antibodies: a Laboratory Manual, page 726, Cold Spring Harbor Pub., 1988). Briefly, monoclonal antibodies can be obtained by injecting mice with a composition comprising an antigen, verifying the presence of antibody production by removing a serum sample, removing the spleen to obtain B lymphocytes, fusing the B lymphocytes with myeloma cells to produce hybridomas, cloning the hybridomas, selecting positive clones that produce antibodies to the antigen, and isolating the antibodies from the hybridoma cultures. Monoclonal antibodies can be isolated and purified from hybridoma cultures by a variety of well-established techniques. Such isolation techniques include affinity chromatography with Protein-A Sepharose, size-exclusion chromatography, and ion-exchange chromatography (see, e.g., Coligan et al., sections 2.7.1-2.7.12 and sections 2.9.1-2.9.3; Barnes et al., Purification of Immunoglobulin G (IgG), in: Methods in Molecular Biology, Vol. 10, pages 79-104, Humana Press, 1992).

An antibody that specifically binds CCTα can be derived from a humanized monoclonal antibody. Humanized monoclonal antibodies are produced by transferring mouse complementarity determining regions from heavy and light variable chains of the mouse immunoglobulin into a human variable domain, and then substituting human residues in the framework regions of the murine counterparts. General techniques for cloning murine immunoglobulin variable domains are described, for example, by Orlandi et al., Proc. Natl. Acad. Sci. U.S.A. 86:3833, 1989. Techniques for producing humanized monoclonal antibodies are described, for example, by Jones et al., Nature 321:522, 1986; Riechmann et al., Nature 332:323, 1988; Verhoeyen et al., Science 239:1534, 1988; Carter et al., Proc Natl Acad Sci USA 89:4285, 1992; Sandhu, Crit. Rev Biotech 12:437, 1992; and Singer et al., J Immunol 150:2844, 1993.

Antibodies can be derived from human antibody fragments isolated from a combinatorial immunoglobulin library (see, for example, Barbas et al., in: Methods: a Companion to Methods in Enzymology, Vol. 2, page 119, 1991; Winter et al., Ann. Rev. Immunol. 12:433, 1994). Cloning and expression vectors that are useful for producing a human immunoglobulin phage library can be obtained, for example, from Stratagene Cloning Systems (La Jolla, Calif.).

In addition, antibodies can be derived from a human monoclonal antibody. Such antibodies are obtained from transgenic mice that have been “engineered” to produce specific human antibodies in response to antigenic challenge. In this technique, elements of the human heavy and light chain loci are introduced into strains of mice derived from embryonic stem cell lines that contain targeted disruptions of the endogenous heavy and light chain loci. The transgenic mice can synthesize human antibodies specific for human antigens, and the mice can be used to produce human antibody-secreting hybridomas. Methods for obtaining human antibodies from transgenic mice are described by Green et al., Nature Genet. 7:13, 1994; Lonberg et al., Nature 368:856, 1994; and Taylor et al., Int Immunol 6:579, 1994.

Antibodies include intact molecules as well as fragments thereof, such as Fab, F(ab′)₂, and Fv which are capable of binding the epitopic determinant. These antibody fragments retain some ability to selectively bind with their antigen or receptor and are defined as follows:

(1) Fab, the fragment which contains a monovalent antigen-binding fragment of an antibody molecule, can be produced by digestion of whole antibody with the enzyme papain to yield an intact light chain and a portion of one heavy chain;

(2) Fab′, the fragment of an antibody molecule can be obtained by treating whole antibody with pepsin, followed by reduction, to yield an intact light chain and a portion of the heavy chain; two Fab′ fragments are obtained per antibody molecule;

(3) (Fab′)₂, the fragment of the antibody that can be obtained by treating whole antibody with the enzyme pepsin without subsequent reduction; F(ab′)₂ is a dimer of two Fab′ fragments held together by two disulfide bonds;

(4) Fv, defined as a genetically engineered fragment containing the variable region of the light chain and the variable region of the heavy chain expressed as two chains; and

(5) Single chain antibody, defined as a genetically engineered molecule containing the variable region of the light chain, the variable region of the heavy chain, linked by a suitable polypeptide linker as a genetically fused single chain molecule.

Methods of making these fragments are known in the art (see for example, Harlow and Lane, Antibodies: A Laboratory Manual, Cold Spring Harbor Laboratory, New York, 1988). An epitope is any antigenic determinant on an antigen to which the paratope of an antibody binds. Epitopic determinants usually consist of chemically active surface groupings of molecules such as amino acids or sugar side chains and usually have specific three dimensional structural characteristics, as well as specific charge characteristics.

Antibody fragments can be prepared by proteolytic hydrolysis of the antibody or by expression in E. coli of DNA encoding the fragment. Antibody fragments can be obtained by pepsin or papain digestion of whole antibodies by conventional methods. For example, antibody fragments can be produced by enzymatic cleavage of antibodies with pepsin to provide a 5S fragment denoted F(ab′)₂. This fragment can be further cleaved using a thiol reducing agent, and optionally a blocking group for the sulfhydryl groups resulting from cleavage of disulfide linkages, to produce 3.5S Fab′ monovalent fragments. Alternatively, an enzymatic cleavage using pepsin produces two monovalent Fab′ fragments and an Fc fragment directly (see U.S. Pat. No. 4,036,945 and U.S. Pat. No. 4,331,647, and references contained therein; Nisonhoff et al., Arch Biochem Biophys 89:230, 1960; Porter, Biochem J 73:119, 1959; Edelman et al., Methods in Enzymology, Vol. 1, page 422, Academic Press, 1967; and Coligan et al. at sections 2.8.1-2.8.10 and 2.10.1-2.10.4).

D. Output of CCTα Expression Data

Any suitable output device or format can be used to transmit the information obtained from the technique used to detect expression of CCTα. For example, the output device can be a visual output device, such as a computer screen, a printed piece of paper or a written piece of paper. In other examples, the output device can be an auditory output device, such as a speaker. In other examples, the output device is a printer. In some cases, the data is recorded in a patient's electronic medical record. In some embodiments, the results of the test used to evaluate gene expression are provided to a user (such as a clinician or other health care worker, laboratory personnel, or patient) in a perceivable output that provides information about the results of the test. In some examples, the output is communicated to the user, for example by providing an output via physical, audible or electronic means (for example, by mail, telephone, facsimile transmission, e-mail or communication to an electronic medical record).

In some examples, the output is accompanied by guidelines for interpreting the data, for example, an indication of the likelihood of diagnosis of cancer. The guidelines need not specify whether cancer is present or absent, although it may include such a diagnosis. In other examples, the output can provide a recommended therapeutic regimen. For instance, based CCTα expression levels, the output can recommend treatment with a genotoxic agent. In some examples, the test may include determination of other clinical information (such as determining the presence or absence of expression of other cancer biomarkers).

### VI. Genotoxic Therapy

In some embodiments of the methods discussed herein, detecting expression of CCTα can be used to determine the response of a subject to genotoxic therapy. In some embodiments, the genotoxic therapy comprises administration of a chemotherapeutic agent. In other embodiments, the genotoxic therapy comprises radiation therapy. Exemplary chemotherapeutic agents and methods of radiation therapy are well known in the art, some of which are described below.

A. Chemotherapy

Chemotherapy is the treatment of cancer with drugs that can destroy cancer cells. The term “chemotherapy” generally refers to cytotoxic or cytostatic drugs which affect rapidly dividing cells in general. Chemotherapy drugs interfere with cell division in various possible ways, e.g. blocking the duplication of DNA, causing DNA strand breaks, or preventing the separation of existing DNA strands or newly formed chromosomes. Most forms of chemotherapy target all rapidly dividing cells and are not specific to cancer cells, although some degree of specificity may come from the inability of many cancer cells to repair DNA damage, while normal cells generally can. Hence, chemotherapy has the potential to harm healthy tissue, especially those tissues that have a high replacement rate (e.g. intestinal lining). These cells usually repair themselves after chemotherapy.

Examples of some of the most commonly used chemotherapy drugs include adriamycin, alkeran, Ara-C, BiCNU, busulfan, CCNU, carboplatinum, cisplatinum, cytoxan, daunorubicin, DTIC, 5-FU, fludarabine, hydrea, idarubicin, ifosfamide, methotrexate, mithramycin, mitomycin, mitoxantrone, nitrogen mustard, taxol (or other taxanes, such as docetaxel), velban, vincristine, VP-16, while some more newer drugs include gemcitabine (Gemzar™), Herceptin™, irinotecan (Camptosar™, CPT-11), leustatin, navelbine, Rituxan™, STI-571, Taxotere™, topotecan (Hycamtin™), Xeloda™ (capecitabine), zevelin and calcitriol.

Chemotherapeutic agents include, for example, alkylating agents, crosslinking agents, antimetabolites, natural products, topoisomerase inhibitors, antineoplastic agents, other miscellaneous agents, or any combination of these. As will be understood to one of skill in the art, some chemotherapeutic agents fall within more than one of the above-listed categories.

Examples of alkylating agents include, but are not limited to, nitrogen mustards (such as mechlorethamine, cyclophosphamide, melphalan, uracil mustard, ifosfamide, or chlorambucil), alkyl sulfonates (such as busulfan), nitrosoureas (such as carmustine, lomustine, semustine, streptozocin, or dacarbazine), and platinum-based drugs (such as ciplastin, carboplatin, or oxaliplatin).

Examples of antimetabolites include, but are not limited to, folic acid analogs (such as methotrexate), pyrimidine analogs (such as 5-FU or cytarabine), and purine analogs, such as mercaptopurine, thioguanine or azathioprine.

Examples of natural products include, but are not limited to, vinca alkaloids (such as vinblastine, vincristine, vinorelbine, or vindesine), epipodophyllotoxins (such as etoposide or teniposide), antibiotics (such as dactinomycin, daunorubicin, doxorubicin, bleomycin, plicamycin, or mitomycin C), and enzymes (such as L-asparaginase).

Examples of miscellaneous chemotherapeutic agents include, but are not limited to, platinum coordination complexes (such as cis-diamine-dichloroplatinum II also known as cisplatin), substituted ureas (such as hydroxyurea), methyl hydrazine derivatives (such as procarbazine), and adrenocrotical suppressants (such as mitotane and aminoglutethimide).

Examples of topoisomerase inhibitors include, but are not limited to, type I topoisomerase inhibitors, such as camptothecins (e.g. irinotecan and topotecan) and type II topoisomerase inhibitors, such as amsacrine, etoposide, etoposide phosphate, and teniposide (which are semisynthetic derivatives of epipodophyllotoxins).

Antineoplastics include, for example, the immunosuppressant dactinomycin, doxorubicin, epirubicin and bleomycin.

B. Radiation Therapy

Radiation therapy is also referred to as radiotherapy, X-ray therapy or irradiation. Radiation therapy involves the use of ionization radiation to kill cancer cells and shrink tumors. Radiation therapy can be administered externally via external beam radiotherapy (EBRT) or internally via brachytherapy. The effects of radiation therapy are localized and confined to the region being treated. Radiation therapy injures or destroys cells in the area being treated (the “target tissue”) by damaging their genetic material, making it impossible for these cells to continue to grow and divide. Although radiation damages both cancer cells and normal cells, most normal cells can recover from the effects of radiation and function properly. The goal of radiation therapy is to damage as many cancer cells as possible, while limiting harm to nearby healthy tissue.

Radiation therapy may be used to treat almost every type of solid tumor, including cancers of the brain, breast, cervix, larynx, lung, pancreas, prostate, skin, stomach, uterus, or soft tissue sarcomas. Radiation is also used to treat leukemia and lymphoma. Radiation dose to each site depends on a number of factors, including the radiosensitivity of each cancer type and whether there are tissues and organs nearby that may be damaged by radiation.

The following examples are provided to illustrate certain particular features and/or embodiments. These examples should not be construed to limit the disclosure to the particular features or embodiments described.

### EXAMPLES

### Example 1

### Identification of CCTα as a Biomarker for Cancer Prognosis

This example describes the identification of CCTα as a second protein recognized by the 8F1 antibody, an antibody raised against the DNA repair protein ERCC1.

### Material and Methods

### Antibodies and Chemicals

Antibodies used were anti-ERCC1 8F1, D-10, FL297 (Santa Cruz Biotechnology), EP2143Y (Abcam), anti-CCTα/PCYT1α (Sigma-Aldrich), and anti-p-tubulin (Sigma-Aldrich).

### Cell Culture and Plasmids

Ovarian carcinoma A2780 cells and HeLa-S3 cells were cultured in RPMI with 10% FBS. C-terminal GFP tagged human CCTα (Origene) was transfected into HeLa-S3 cells, and GENETICIN™ was used to select stable clones. CCTα was knocked-down with shRNA constructs (Origene). Stable clones were selected with puromycin. ERCC1-deficient control cells are described in Vaezi et al. (Clin Cancer Res 17(16): 5513-5522, 2011).

### Immunoprecipitation

Protein A/G beads were conjugated to 8F1 (non-specific) or D-10 (specific) antibody. A2780 cells were lysed in RIPA buffer with protease inhibitors and incubated with antibody-conjugated beads. The beads were boiled in Laemmeli buffer, and proteins were resolved by SDS-PAGE. The gel was silver-stained (BioRad), and bands were analyzed by mass spectrometry.

### Immunohistochemistry and Quantification

Cell pellets were formalin fixed and paraffin embedded (FFPE). For tumor analysis, tissue microarrays of FFPE tumors and control tissues were used. Each tumor was represented by three distinct cores for NSCLC and two for HNSCC. Tissue microarrays were processed for antigen retrieval and IHC as described elsewhere (Vaezi et al., Clin Cancer Res 17(16): 5513-5522, 2011; Zheng et al., N Engl J Med 356(8): 800-808, 2007). The antibody dilutions used were 1:100 for 8F1, 1:250 for FL297, 1:200 for EP2143Y, and 1:200 for CCTα. Digital image analysis for normalized nuclear signal intensity was quantified by automated quantitative analysis (AQUA) for NSCLC and APEIRO for HNSCC (Vaezi et al., Clin Cancer Res 17(16): 5513-5522, 2011).

### Tumor Samples and Patient Cohorts

The well-characterized, single-institution cohort of 187 early-stage NSCLC treated with curative intent was described elsewhere (Zheng et al., N Engl J Med 356(8): 800-808, 2007). Tumors were collected prospectively. The median follow-up was 54 months (range: 28-139 months). The HNSCC cohort (n=80) is a retrospective, single-institution cohort of representative pretreatment biopsy or resection specimens described elsewhere (Duvvuri et al., Cancer Res 72(13): 3270-3281, 2012). Median follow-up was 41 months (range: 31-178 months). All sites in the aero-digestive tract, stages, and treatments were included.

### Statistical Analysis

The Spearman rank correlation coefficient was used for an overview of bivariate correlations. The association among the signal intensities of the four antibodies was examined by linear regression on antibody 8F1; average intensities between the cores were used. A best fitting linear model was established that included variance-stabilizing transformations, as needed based on review of residuals. Regression slopes and contributions to explained variation were estimated from the regression models. Proportional hazards modes were formulated to estimate the influence of protein expression adjusted for clinical or pathologic factors as needed. Overall survival was defined as time from surgery to death and was censored if the patient was still alive at last follow-up. For analyzing recurrence-free survival in NSCLC, the full cohort was not used to assess time due to a substantial early dropout rate and conflicting definitions of recurrence-free survival event. An index cohort of known outcomes was constructed that included patients who (1) had a documented recurrence within 3 years or (2) survived at least three years without recurrence. This index cohort was used for the study of recurrence-free survival. For the HNSCC cohort, recurrence time was defined as time from treatment to the first evidence of recurrence with censoring for patients who died without recurrence or who had incomplete follow-up. Evidence of association with survival or recurrence and continuously measured covariates was illustrated with a Kaplan Meier plot of the covariate after splitting at the median. A log rank test was included.

### Results

### 8F1 Recognizes CCTα

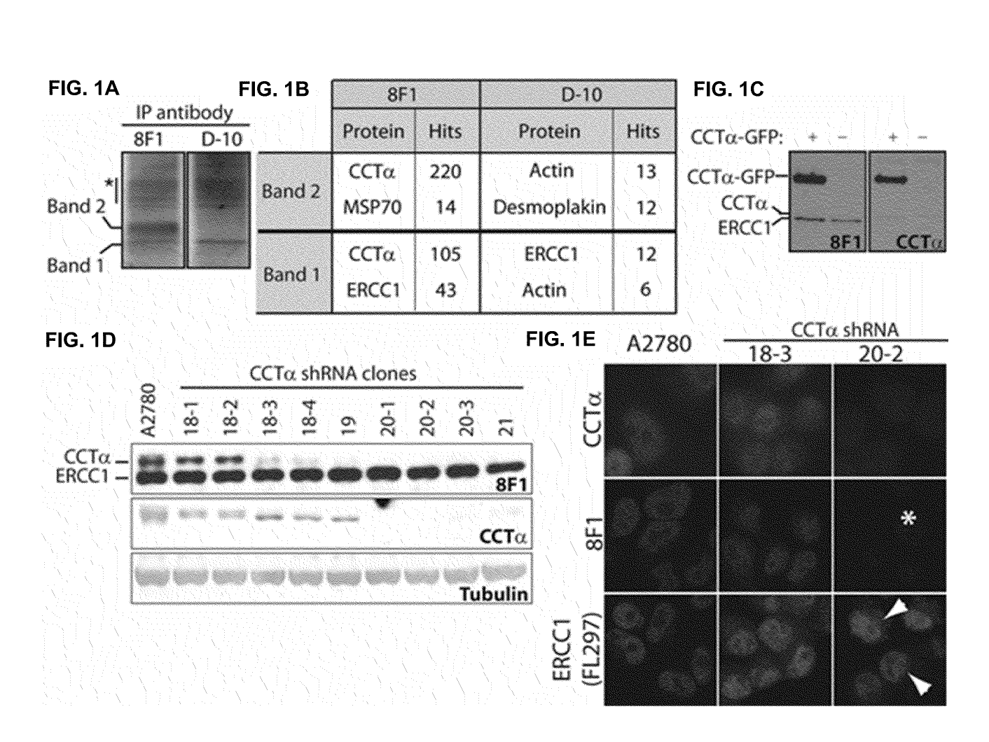

It was previously demonstrated that 8F1 recognizes both ERCC1 and a second, unidentified nuclear protein that may interfere with the measurement of ERCC1 in clinical samples (Niedernhofer et al., N Engl J Med 356(24): 2538-2540; author reply 40-1, 2007). To determine the identity of this protein, whole cell lysates of A2780 ovarian cancer cells were immunoprecipitated using 8F1 or the ERCC1-specific antibody clone D-10 (Bhagwat et al., Cancer Res 69(17): 6831-6838, 2009) and proteins were separated by electrophoresis (FIG. 1A). Both antibodies pulled down ERCC1. The 8F1 antibody also precipitated a second, slower migrating protein, which could be seen by silver stain (FIG. 1A). Mass spectrometry identified the band as CCTα, a lipid-modifying enzyme involved in phosphatidyl choline synthesis (FIG. 1B) (Jackowski and Fagone, J Biol Chem 280(2): 853-856, 2005; Li and Vance, J Lipid Res 49(6): 1187-1194, 2008) that localizes to the nucleus (Wang et al., J Biol Chem 268(8): 5899-5904, 1993; DeLong et al., J Biol Chem 275(41): 32325-32330, 2000). The lower band was identified as ERCC1 (see Table 2). As a negative control, the same region of the gel from the D-10 immunoprecipitate yielded no CCTα peptides, but ERCC1 was identified. This result suggests that 8F1, but not D-10, reacts with two distinct immune targets: ERCC1 and CCTα.

Shown in Table 2 are the proteins most frequently identified in each sample, ranked by the number of hits. The numbers were obtained from two independent mass spectrometry analyses.

TABLE 2
Summary of mass spectrometry results

| Immunoprecipitation with 8F1 | | | Immunoprecipitation with D-10 | | |
|---|---|---|---|---|---|
| | Number | | | Number | |
| | unique of | Number of | Identity of corresponding | unique of | Number of |
| Identity of top band | peptides | hits | area on the gel | peptides | hits |
| Choline-phosphate | 16 | 220 | Actin | 6 | 13 |
| cytidylyltransferase α | | | | | |
| Mitochondrial stress | 8 | 14 | Desmoplakin | 11 | 12 |
| protein p70 | | | | | |
| Homerin | 2 | 11 | Filaggrin-2 | 2 | 9 |
| Actin | 4 | 8 | Plakoglobin | 3 | 7 |
| Dermicidin | 2 | 8 | Mitochondrial stress | 5 | 6 |
| | | | protein p70 | | |
| Desmoplakin | 2 | 8 | Dermicidin | 4 | 5 |
| Filaggrin-2/Titin | 4 | 3 | ATP-dependent RNA helicase | 1 | 4 |
| ERCC1 | 1 | 2 | ERCC1 | 2 | 3 |
| | | | Desmin | 1 | 3 |
| | | | Rab and Ras interactor 2 | 2 | 3 |
| | | | Desmoglein | 2 | 2 |
| | | | Ankyrin | 2 | 2 |
| | | | Kinesin 1 | 1 | 2 |
| | | | p65 | 1 | 2 |

| Immunoprecipitation with 8F1 | | | Immunoprecipitation with D-10 | | |
|---|---|---|---|---|---|
| | Number of | Number of | | Number of | Number of |
| Identity of bottom band | peptides | hits | Identity of band single | peptides | hits |
| Choline-phosphate | 12 | 105 | ERCC1 | 6 | 12 |
| cytidylyltransferase α | | | | | |
| ERCC1 | 5 | 43 | Actin | 1 | 6 |
| Ribonucleoprotein C | 5 | 11 | Lipoamide | 1 | 6 |
| | | | acetyltransferase | | |
| Dermcidin | 2 | 10 | Mitochondrial | 3 | 4 |
| | | | stress p70 | | |
| Mitochondrial | 3 | 5 | Homerin | 1 | 2 |
| stress p70 | | | | | |
| Actin | 3 | 4 | Nucleophosmin | 1 | 2 |
| Nucleophosmin | 1 | 2 | Ribonucleoprotein | 1 | 2 |
| | | | C-like | | |
| P65 | 1 | 2 | | | |

It was further confirmed that 8F1 reacted with CCTα by stable expression of GFP-tagged CCTα in HeLa cells, which expressed only trace amounts of endogenous CCTα c. Recombinant CCTα, which migrates more slowly than the endogenous protein due to the GFP-tag, was readily detected by 8F1 (FIG. 1C, left panel). The identity was confirmed with a commercially available antibody recognizing CCTα (FIG. 1C, right panel).

To determine the degree to which CCTα contributes to 8F1s nuclear signal, CCTα was knocked down in A2780 cells using shRNA, which led to disappearance of the band corresponding to CCTα while ERCC1 levels remained unchanged (FIG. 1D). Individual stable clones, with varying levels of CCTα expression were then evaluated by immunofluorescence using 8F1 and the anti-CCTα antibody. 8F1 nuclear signal was dramatically reduced in clones with reduced CCTα expression (FIG. 1E), while ERCC1 expression assessed by the ERCC1-specific antibody FL297 remained unchanged (FIG. 1E). These results indicate that, in vitro, 8F1 nuclear signal varied as a function of CCTα expression, even when ERCC1 expression remained constant. This demonstrates that 8F1 has two distinct immune targets: ERCC1 and CCTα. Both localize to the nucleus, interfering with the measurement of ERCC1 expression when 8F1 is used.

### 8F1 Signal Intensity Depends on ERCC1 and CCTα Expression Levels in NSCLC

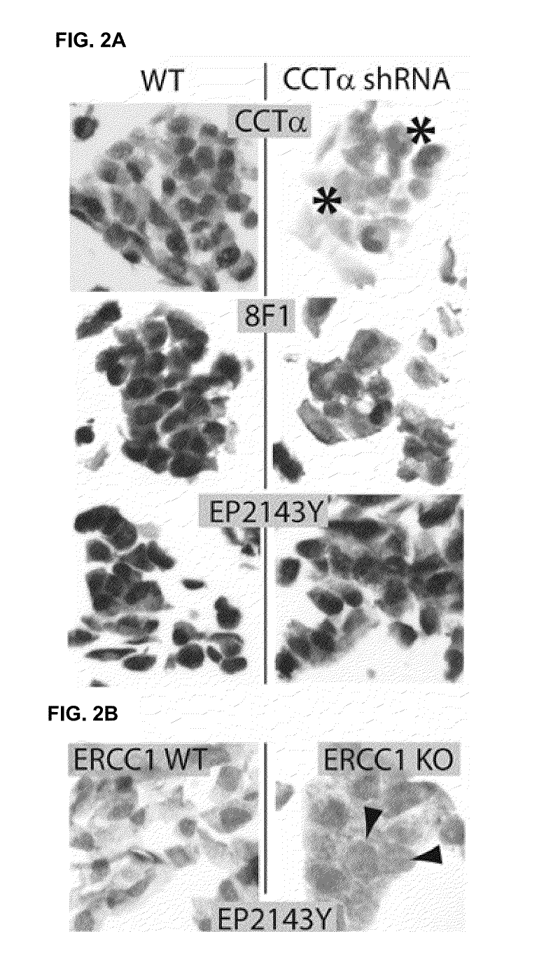

It was next evaluated whether the in vitro findings are relevant for quantification of ERCC1 expression by IHC. Using knock-down cell lines, commercial antibodies raised against CCTα or ERCC1 were characterized to determine their specificity and suitability for IHC. Only background nuclear staining was observed in cells in which the respective protein had been substantially knocked down, while signal was readily visible in the control cells (FIG. 2A and FIG. 2B, respectively). By IHC with an ERCC1-specific antibody (EP2143Y), the nuclear signal was unchanged in CCTα knock-down cells (FIG. 2A), but dramatically decreased in ERCC1-deficient cells (FIG. 2B). In contrast, when 8F1 was used, the nuclear signal decreased in CCTα knock-down cells (FIG. 2A). These results show that the antibodies were specific for their respective antigens, that reduced CCTα levels do not affect ERCC1 expression, and that the 8F1 signal is a result of detecting both ERCC1 and CCTα.

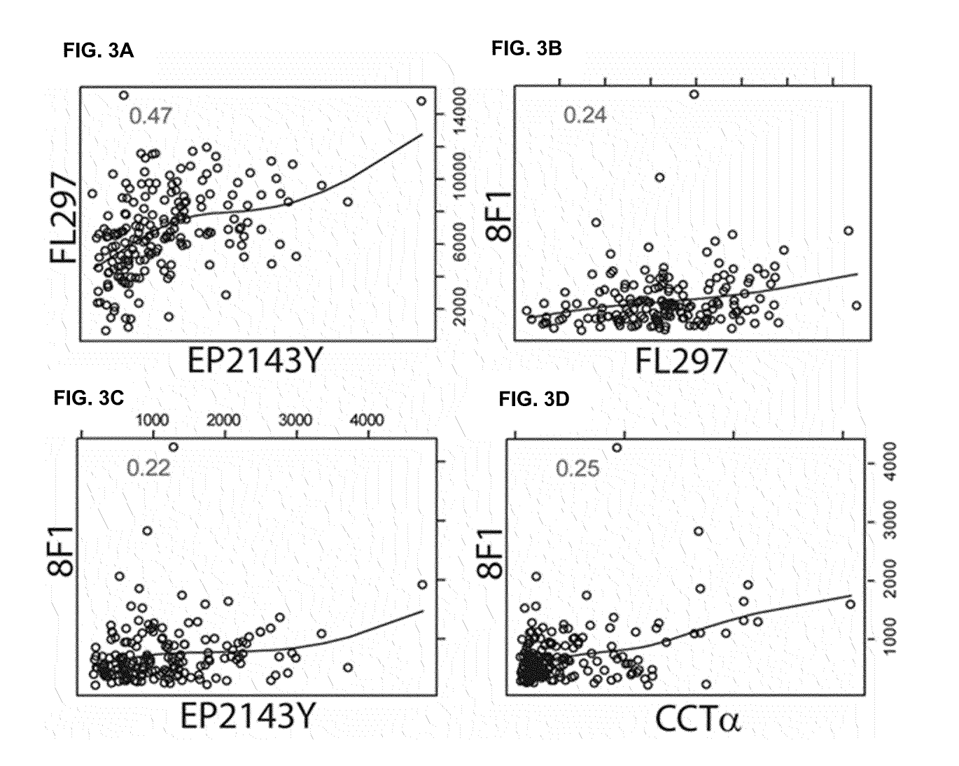

Since 8F1 is thought to be a biomarker of ERCC1 expression in NSCLC, the extent to which 8F1 signal intensity estimated ERCC1 protein expression was examined by IHC. This analysis used a well-characterized cohort of early stage NSCLC treated by surgery alone (n=187) that was previously used to demonstrate a significant correlation between high 8F1 signal and improved patient survival (Zheng et al., N Engl Med 356(8): 800-808, 2007). AQUA was performed on samples stained with specific anti-ERCC1 antibodies (FL297 or EP2143Y) or 8F1, and signal intensities were compared. There was a strong correlation between the signal intensities of the two ERCC1-specific antibodies (FL297 or EP2143Y; rho r=0.47, p=<0•0001) (FIG. 3A). However, the 8F1 signal had a weaker correlation with the signal from either EP2143Y (rho=0•22, p=0•0037) or FL297 (rho=0•24, p=0•0012 FIGS. 3B-3C). There was also a significant, equally weak, correlation between 8F1 and CCTα signal (rho=0.25; p=0.0008) (FIG. 3D).

The relative contribution of ERCC1 and CCTα to the variation of 8F1 was determined by linear regression on 8F1 signal. It was found that CCTα and to a lesser degree ERCC1 (FL297 but not EP2143Y) were determinants of the 8F1 signal (Table 3). This result supports the conclusion that 8F1 nuclear signal is strongly influenced by the newly identified 8F1 antigen CCTα in early stage NSCLC.

TABLE 3
Contribution of ERCC1 and CCTα to 8F1 staining

| | | | |
|---|---|---|---|
| | Estimate | | |
| Antibody* | (Std. Error) | T statistic | p |

NSCLC

| | | | |
|---|---|---|---|
| FL297 | 0·183 (0·077) | 2·371 | 0·019 |
| EP2143Y | 0·071 (0·067) | 1·052 | 0·294 |
| CCTα in AC | 0·006 (0·064) | 0·102 | 0·919 |
| CCTα in LCC | 0·183 (0·173) | 1·055 | 0·293 |
| CCTα in SCC | 0·276 (0·074) | 3·754 | <0·0001 |

HNSCC

| | | | |
|---|---|---|---|
| EP2143Y | 0·01 (0·014) | 1·09 | 0·283 |
| CCTα | 0·034 (0·005) | 6·43 | <0·0001 |

*Log transformation

### CCTα Expression but not ERCC1 Depends on Tumor Histology

The NSCLC cohort contains multiple histological tumor types (squamous cell (SCC), large cell (LCC), adenocarcinoma (AC)), tumor stages (American Joint Committee on Cancer stage 1A and 1B), and patient characteristics. Tests for differences in expression revealed that CCTα was differentially expressed by histological subtype (p<0•0001) with higher levels in SCC than in AC or LCC (Table 4). Tumor stage was not associated with CCTα expression level (Table 4).

TABLE 4
Distribution of Antibody Expression Levels by Stage and Histology in
NSCLC
Medians (interquartile range) and p value for test of equality

| | Histology | | | | T stage | | |
|---|---|---|---|---|---|---|---|
| Antibody | AC | SCC | LCC | p¹ | Stage 1 | Stage 2 | p ² |
| 8F1 | 582 | 629 | 591 | 0·325 | 670 | 565 | 0·201 |
| | (393-870) | (436-945) | (475-781 | | (403-991) | (412-708) | |
| EP2143Y | 872 | 922 | 1317 | 0·055 | 941 | 940 | 0·720 |
| | (553-1375) | (576-1425) | (783-287) | | (596-1382) | (580-1450) | |
| FL297 | 6332 | 6893 | 7120 | 0·089 | 6404 | 6730 | 0·341 |
| | (4608-7740) | (5425-8983) | (5541-8529) | | (4714-8233) | (5095-8649) | |
| CCTα | 954 | 2393 | 1361 | <0·001 | 1212 | 1567 | 0·152 |
| | (628-2256) | (1135-4775) | (1042-1916) | | (676-2915) | (855-3373) | |

¹Kruskal-Wallis test p;
² Wilcoxon test p

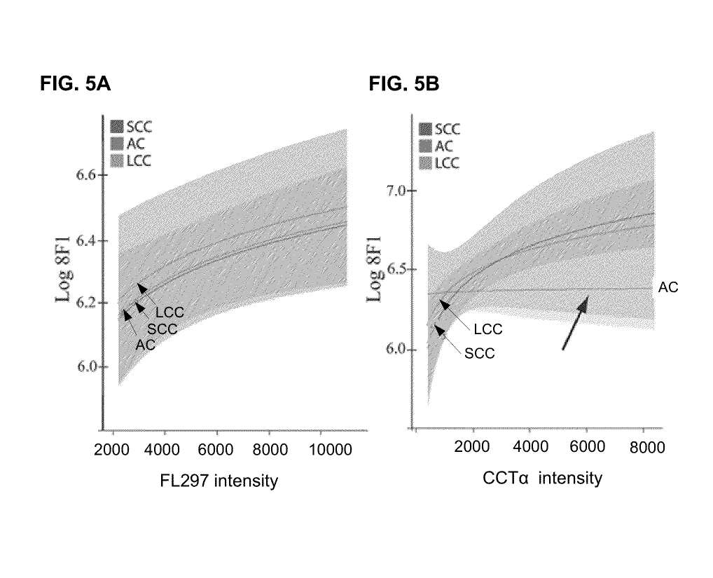

The relative contribution of CCTα to the 8F1 signal was stratified by tumor histology. CCTα was a significant contributor only for SCC (slope=0•282, p=0•0001) (Table 3 and FIG. 5). In contrast, FL297 contributed to 8F1 signal across all histologic types of NSCLC. This suggests that the influence of CCTα expression on 8F1 signal intensity is dependent on the tumor histology.

### In HNSCC, the 8F1 Signal is Primarily a Result of CCTα

The association between 8F1 signals and ERCC1 (EP2143Y) and CCTα expression levels was evaluated in a cohort of 60 HNSCC (characteristics summarized in Table 5) (Duvvuri et al., Cancer Res 72(13): 3270-3281, 2012). In a linear regression model, CCTα was strongly correlated with 8F1 (p<0•0001), but ERCC1 was not (p=0•283) (Table 3). These findings support the notion that CCTα expression is a major contributor to 8F1 immunostaining in both HNSCC and NSCLC.

TABLE 5
HNSCC cohort characterization

| Characteristic | Subcategory | n | (%) |
|---|---|---|---|
| Patients | | 60 | |
| Mean age | | 58.1 | |
| | <60 yrs | 30 | (50) |
| | 60 or older | 30 | (50) |
| Gender | M | 42 | (70) |
| | F | 18 | (30) |
| Smoker | yes | 48 | (80) |
| | no | 12 | (20) |
| Tumor site | oral cavity | 25 | (42) |
| | oropharynx | 13 | (22) |
| | larynx | 17 | (28) |
| | hypopharynx | 1 | (2) |
| | unknown primary | 3 | (5) |
| | other | 1 | (2) |
| Stage | I | 3 | (5) |
| | II | 10 | (17) |
| | III | 8 | (13) |
| | IV | 21 | (48) |
| | n/a | 10 | (17) |
| Treatment | Surgery (curative intent) | 50 | (83) |
| | XRT (adjuvant or primary) | 30 | (50) |
| | XRT unknown | 4 | (7%) |
| Mean follow-up | (days) | 1499 | 1190-1807* |

*95% confidence interval

### Correlation Between CCTα Expression and Clinical Outcomes in NSCLC and HNSCC

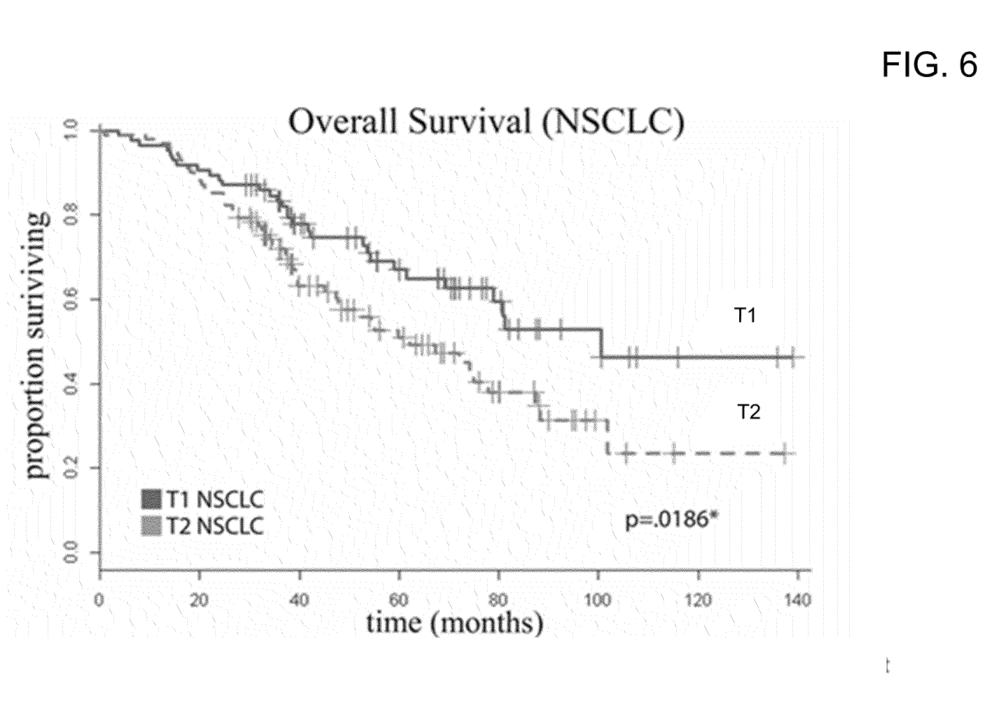

Based on numerous prior clinical studies using 8F1 (Roth and Carlson, Clin Lung Cancer 12(6): 393-401, 2011; Vaezi et al., Pharmgenomics Pers Med 4: 47-63, 2011), the influence of CCTα on 8F1 staining leads to the possibility that CCTα expression level may have intrinsic value as a biomarker. To address this, the correlation between CCTα protein expression and clinical outcomes was evaluated in a cohorts in which 8F1 was previously discovered to have significant predictive value. Critical evaluation of these cohorts revealed that overall survival was a reliable endpoint, with data similar to what is expected in early lung cancer (FIG. 6). However, a high number of patients were lost to follow up within the first 3 years. Therefore, an indicator cohort of 86 patients was established using only unflawed data in which recurrence-free survival can be reported with confidence.

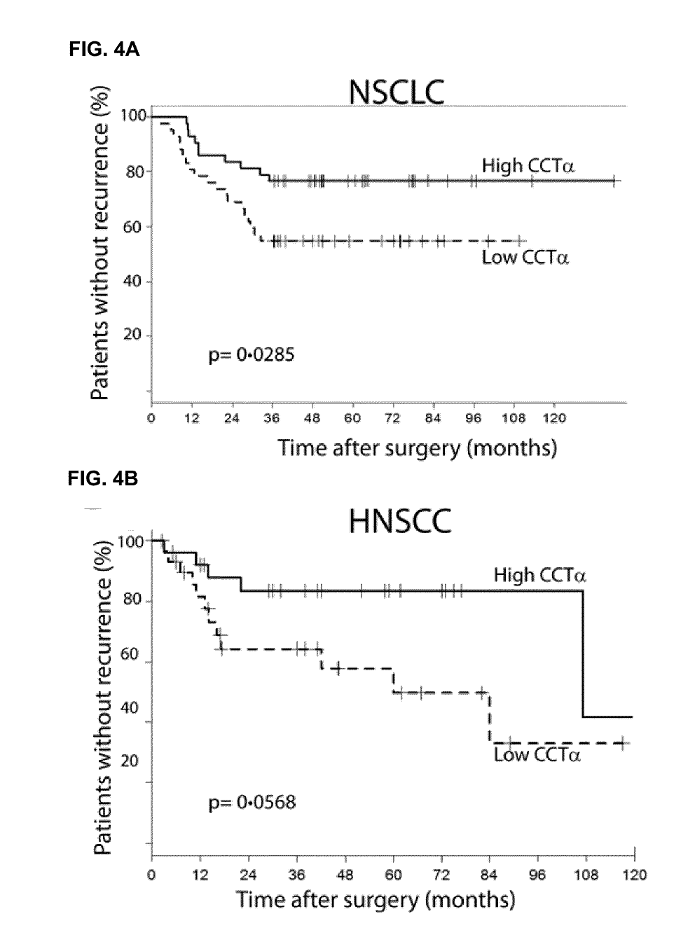

In a univariate analysis, there was no association between ERCC1 level measured with either of the ERCC1-specific antibodies and recurrence-free survival (Table 6) or overall survival (Table 7). However, CCTα expression trended to associate with recurrence-free survival (HR=0•75, CI 0•51-1.15, p=0•185) and overall survival (HR 0•84, CI 0•67-1.05, p=0•125). Furthermore, Kaplan-Meier survival estimates based on splitting CCTα levels at the median showed that patients with high CCTα expression had a greater three year recurrence-free survival (0•77 for high vs. 0•55 years for low expressers, p=0•029) (FIG. 4A). These results provide the first evidence that CCTα has potential value as a biomarker in solid tumors.

TABLE 6
Proportional Hazards Models for Disease-Free Survival in NSCLC (n = 86)

| | | Univariate | | | Multivariate | | |
|---|---|---|---|---|---|---|---|
| Covariate | Reference | HR | 95% CI | p | HR | 95% CI | p |
| 8F1 | 407-908 | 0·98 | 0·70-1·38 | 0·908 | | | |
| CCTα | 742-3039 | 0·75 | 0·50-1·15 | 0·185 | 0·93 | 0·60-1·44 | 0·754 |
| EP2143Y | 582-1425 | 0·87 | 0·58-1·31 | 0·516 | | | |
| FL297 | 4918-8528 | 0·75 | 0·43-1·31 | 0·319 | | | |
| Histology: LCC | AC | 2·23 | 0·97-5·10 | 0·058 | 2·03 | 0·80-5·16 | 0·095 |
| Histology: SCC | AC | 0·56 | 0·22-1·45 | 0·233 | 0·72 | 0·26-2·01 | 0·527 |
| Age | 65-76 | 0·89 | 0·57-1·39 | 0·611 | | | |
| T stage 2 | Stage 1 | 1·84 | 0·89-3·83 | 0·102 | | | |
| Performance Status 1 | Status 0 | 2·12 | 0·98-4·59 | 0·050 | 2·12 | 0·91-4·980 | 0·080 |
| Gender: Male | Female | 0·72 | 0·35-1·49 | 0·377 | | | |
| Pack Years | 35-75 | 1·31 | 1·0-1·72 | 0·049 | 1·33 | 0·98-1·81 | 0·067 |

TABLE 7
Proportional Hazards Models for Overall Survival (N = 187)

| | | Univariate | | | Multivariate | | |
|---|---|---|---|---|---|---|---|
| Covariate | Reference | HR | 95% CI | p | HR | 95% CI | p |
| 8F1 | 407-900 | 0·94 | 0·75-1·18 | 0·590 | | | |
| CCTα | 742-3093 | 0·84 | 0·67-1·05 | 0·125 | 0·88 | 0·69-1·11 | 0·278 |
| EP2143Y | 582-1425 | 1·10 | 0·80-1·27 | 0·950 | | | |
| FL297 | 4918-3610 | 0·88 | 0·66-1·18 | 0·398 | | | |
| Histology: LCC | AC | 1·94 | 1·07-3·51 | 0·029 | 1·66 | 0·89-3·11 | 0·110 |
| Histology: SCC | AC | 0·91 | 0·56-1·49 | 0·705 | 0·91 | 0·53-1·56 | 0·723 |
| Age | 65-76 | 1·12 | 0·84-1·50 | 0·430 | | | |
| T stage 2 | Stage 1 | 1·71 | 1·09-2·68 | 0·019 | 1·63 | 1·02-2·62 | 0·043 |
| Performance Status 1 | Status 0 | 1·67 | 1·03-2·69 | 0·036 | 1·62 | 0·98-2·68 | 0·060 |
| Gender: Male | Female | 1·39 | 0·89-2·15 | 0·145 | | | |
| Pack Years | 35-75 | 1·17 | 0·96-1·44 | 0·118 | | | |

To test whether these results apply to HNSCC, the association between CCTα and survival was evaluated in a cohort of 60 patients. Using a proportional hazard regression model, an association between CCTα level (p=0•046), but not 8F1 (p=0•309) or EP2143Y (p=0•415), and recurrence-free survival was identified. Patients with a high CCTα level compared to low CCTα level had a longer survival (p=0•057), with a survival curve separation reminiscent of the NSCLC data (FIG. 4B). Taken together, these observations indicate that CCTα can be used as a biomarker in solid tumors of the upper aero-digestive tract.

In view of the many possible embodiments to which the principles of the disclosed invention may be applied, it should be recognized that the illustrated embodiments are only preferred examples of the invention and should not be taken as limiting the scope of the invention. Rather, the scope of the invention is defined by the following claims. We therefore claim as our invention all that comes within the scope and spirit of these claims.

1. A method of determining the prognosis of a patient with cancer, or predicting the response of a cancer patient to treatment with a genotoxic therapy, comprising specifically detecting expression of choline phosphate cytidylyltransferase-α (CCTα) in a sample obtained from the subject, wherein an increase in expression of CCTα in the sample compared to a control indicates a good prognosis for the patient, or predicts a poor response to the genotoxic agent.

2. The method of claim 1, wherein specifically detecting expression of CCTα in a sample comprises detecting expression of CCTα without detecting expression of excision repair cross-complementation group 1 (ERCC1).

3. The method of claim 1, wherein the good prognosis comprises an increase in the likelihood of recurrence-free survival.

4. The method of claim 1, wherein detecting expression of CCTα comprises detecting CCTα protein.

5. The method of claim 1, wherein detecting expression of CCTα comprises detecting CCTα mRNA.

6. The method of claim 1, wherein detecting expression of CCTα comprises:
(i) detecting increased expression of CCTα by at least 2-fold, at least 3-fold, at least 4-fold or at least 5-fold relative to the control;
(ii) detecting increased expression of CCTα mRNA by at least 5-fold, at least 10-fold, at least 25-fold or at least 50-fold relative to the control; or
(iii) both (i) and (ii).

7. The method of claim 1, wherein the control is a non-tumor sample obtained from the subject or a healthy subject.

8. The method of claim 1, wherein the control is a reference value.

9. The method of claim 1, wherein the sample is a tumor sample.

10. The method of claim 1, wherein the cancer is a solid tumor.

11. The method of claim 10, wherein the solid tumor is non-small cell lung carcinoma, gastric carcinoma, esophageal carcinoma, pancreatic cancer, colon cancer, breast cancer, brain cancer, head and neck squamous cell carcinoma, squamous cell carcinoma of the lung, pulmonary papillary adenocarcinoma, mesothelioma, esophageal cancer, nasopharyngeal cancer, prostate cancer, adrenocortical carcinoma, cutaneous neuroendocrine cancer, gallbladder cancer, bile duct cancer, cervical cancer, serous ovarian cancer, epithelial ovarian cancer, endometrial cancer, bladder cancer, urothelial carcinoma, Ewing sarcoma, testicular cancer, neuroendocrine cancer, liver cancer, hepatocellular carcinoma, pituitary cancer or glioma.

12. The method of claim 1, wherein the cancer is a hematologic cancer.

13. The method of claim 12, wherein the hematologic cancer is myeloid leukemia, acute lymphoblastic leukemia, marginal zone B cell lymphoma, acute lymphoblastic anemia, acute lymphocytic leukemia, lymphoma or thrombocythemia.

14. The method of claim 1, wherein the genotoxic therapy comprises radiation therapy.

15. The method of claim 1, wherein the genotoxic therapy comprises administration of a genotoxic agent.

16. The method of claim 15, wherein the genotoxic agent is a chemotherapeutic agent.

17. The method of claim 16, wherein the chemotherapeutic agent is a platinum-based chemotherapeutic agent.

18. The method of claim 17, wherein the platinum-based chemotherapeutic agent is cisplatin, carboplatin or oxaliplatin.

19. The method of claim 16, wherein the chemotherapeutic agent is an alkylating agent.

20. The method of claim 19, wherein the alkylating agent is BCNU, cyclophosphamide, melphalan, mitomycin C, mechlorethamine or a psoralen.

21. The method of claim 16, wherein the chemotherapeutic agent is bleomycin, doxorubicin or etoposide.

22. The method of claim 1, further comprising administering an appropriate therapy to the patient with cancer.

23. The method of claim 22, wherein the appropriate therapy comprises genotoxic therapy if a decrease in expression of CCTα is detected in the sample compared to a control.
